(19) United States
(12) Patent Application Publication
(10) Pub. No.: US 2023/0349002 A1
(43) Pub. Date: Nov. 2, 2023

(54) METHOD AND COMPOSITIONS FOR DRUG RESISTANCE SCREENING

(71) Applicant: QUADRAM INSTITUTE BIOSCIENCE, Norfolk (GB)

(72) Inventors: Justin Joseph O’GRADY, Norfolk (GB); Gemma Louise KAY, Norfolk (GB); Michael John STRINDEN, Norfolk (GB)

(21) Appl. No.: 18/044,055

(22) PCT Filed: Aug. 16, 2021
(86) PCT No.: PCT/GB2021/052121

§ 371 Date: Mar. 3, 2023

(30) Foreign Application Priority Data
Sep. 4, 2020 (GB) 2013928.3

(51) Int. Cl. C12Q 1/6844; C12Q 1/689

(52) U.S. Cl. CPC C12Q 1/689; C12Q 1/6844; C12Q 2600/106; C12Q 2600/156; C12Q 2600/16

(57) ABSTRACT

The disclosure relates to novel primers, and their use to detect the presence of drug resistance mutations in a sample from a subject with suspected or confirmed Tuberculosis.

### CROSS-REFERENCE TO RELATED APPLICATIONS

The present application is a filing under 35 U.S.C. 371 as the National Stage of International Application No. PCT/GB2021/052121, filed Aug. 16, 2021, entitled “METHOD AND COMPOSITIONS FOR DRUG RESISTANCE SCREENING,” which claims priority to United Kingdom Application No. 2013928.3filed with the Intellectual Property Office of the United Kingdom on Sep. 4, 2020, both of which are incorporated herein by reference in their entirety for all purposes.

### INCORPORATION BY REFERENCE OF MATERIAL IN ASCII TEXT FILE

This application incorporates by reference the Sequence Listing contained in the following ASCII text file being submitted concurrently herewith:

File name: 4906-00100 1811067PPUS Sequence Listing; created on Feb. 15, 2023; and having a files size of 7 KB.

The information in the Sequence Listing is incorporated herein in its entirety for all purposes.

### FIELD OF THE INVENTION

The invention to which this application relates is a new diagnostic methodology and primers and/or drug susceptibility testing (DST) assay. In particular, the present invention relates to novel primers, and their use in a method of identifying and/or detecting the presence of drug resistance mutations in a sample from subjects with suspected or confirmed Tuberculosis.

### BACKGROUND

### Mycobacteria and Tuberculosis

Tuberculosis (TB), caused primarily by Mycobacterium tuberculosis^{1,2}, is a disease of global health importance³⁻⁵. Mycobacterium tuberculosis and related bacteria in the Mycobacterium tuberculosis complex (MTBc) emerged at least 11,000 years ago and have been coevolving with their hosts since^{6,7}. This history has resulted in a highly transmissible taxon of bacteria with longevity within their host and advanced methods of immune system evasion⁷.

Due to this coevolution, modern M. tuberculosis and members of the MTBc share numerous characteristics and are found in every known environment (except in the polar regions) along with members of the Non-Tuberculous mycobacterium (NTM) group^{7,8}. The MTBc is made up of 10 mycobacterium capable of causing TB or TB-like disease within their hosts, with the three specialized human TB species being Mycobacterium tuberculosis sensu stricto, Mycobacterium canettis and Mycobacterium africanum^{1,7,9}. Additionally, zoonotic TB transfer is well documented from cattle (Mycobacterium bovis), goats and sheep (Mycobacterium caprae), seals and sea lions (Mycobacterium pinnipedii), and rodents (Mycobacterium microti) into humans and vice versa^{4,6,7}. Recently, three more species have been added; Mycobacterium orygis in cattle and antelope^{7,11}, Mycobacterium suricattae in meerkats^{7,11}, and Mycobacterium mungi in mongeese^{7,12}.

Current research demonstrates MTBc members are highly genetically homogenous with up to 99.7% nucleotide identity and having identical 16S sequences⁷. MTBc members are primarily clonal with little horizontal gene transfer making differentiation between species difficult at the genetic level and impossible using microscopic methods^{2,4,6,13}.

Mycobacteria are gram-positive acid-fast bacilli approximately 2 µm long, which are primarily transmitted via aerosols; they are strictly intracellular, and do not have a known environmental reservoir outside of their endemic hosts^{1,7,14}. Lipid-rich cellular walls and layers of peptidoglycan, lipoglycan, mycolic acids, and waxes create an extremely hardy microbe^{7,14}. A defining characteristic of many mycobacteria, and all members of the MTBc, is fastidiousness and slow rate of growth in culture and in vivo^{2,6,13,16}.

Tuberculosis most commonly presents as a pulmonary disease (around 80% of cases), although extrapulmonary and disseminated disease presentations do also occur^{1,2,17}. Mycobacterial diseases cause a high burden of disease in low- and middle-income and developing countries (LMICs) around the world^{3,6,18}. It is estimated that one-third of the human population harbour latent TB (LTBI) and there are between nine and eleven million incident TB cases annually, according to the World Health Organization (WHO)¹⁹. The number of annual fatalities attributed to TB has been estimated at 1.5-2 million deaths globally, making TB the greatest single threat for infection associated mortality^{6,20,21}.

### Mycobacterial Drug Resistance

The WHO defines drug resistance as a microorganism’s resistance to an antimicrobial drug that was once able to treat an infection by that microorganism. The emergence of drug resistant (DR) strains of TB is largely a result of inconsistent practice of treatment protocols, delayed treatment and/or patients defaulting on lengthy treatment courses, leading to positive selection for drug-resistance and a higher incidence of resistant strain transfer between hosts^{3,22,23}.

There are currently several types of drug-resistant TB: multidrug-resistant (MDR) which is resistant to at least rifampicin and isoniazid; extensively drug-resistant (XDR) which has added resistances to any fluoroquinolone and at least one second-line injectable medication beyond what is found in MDR; extremely drug-resistant (XXDR) which is resistant to all first- and second-line medications; and totally drug-resistant (TDR) which has resistance to all current TB medications^{16,24}. Additionally, some species within the MTBc have lineage specific inherent resistances, e.g. M. bovis and M. canettii, which if misdiagnosed can complicate resistance-control methods^{2,22,24}.

Drug-resistant TB (DR-TB) is a growing issue globally as it increases in incidence^{21,22,25}. Concerns are that drug-resistant strains will reverse the progress made towards TB eradication^{6,22,23}. The incidence of drug resistant-TB worldwide has increased at least 10-fold in the past decade, with only 4.9% of patients demonstrating drug resistance in 2009 compared to 51% in 2018¹⁹. In 2018 nearly 500,000 of approximately 10.5 million TB cases in the world were MDR and of those 31,000 (6.2%) were XDR¹⁹.

MDR-TB is the most common type of resistance^{16,24}. MDR is defined as a TB strain which is resistant to isoniazid and rifampicin²⁵. MDR-TB strains are typically treated with traditional WHO endorsed drug regimens which require a 6-month course of first- and second-line antibiotics. XDR-TB is an MDR strain with additional resistance to the second-line medications of any fluoroquinolones and amikacin, capreomycin, or kanamycin^{25,26}. The specific regimen chosen to treat XDR-TB can be guided by culture or molecular (e.g. GenoType MTBDRsl - Bruker) drug susceptibility testing (DST) assays^{6,26,27}where available. Due to difficulties in diagnosing and treating MDR and XDR strains of TB, the mortality rates in these cases are high with approximately 50% mortality MDR and over 70% in XDR-TB infections ²⁵.

The first line treatment for TB is a combination of antibiotics; rifampicin, isoniazid, ethambutol, and pyrazinamide over 6 months. Resistance to these antibiotic therapies leads to the use of second-line antibiotics (fluoroquinolones, amikacin, capreomycin, and kanamycin), which are less effective and more toxic^{24,25}. These therapeutics often require injections which necessitate more advanced medical infrastructure and oversight for treatment²⁴.

Drug resistance in Mycobacteria is mutational, rather than transferrable, and numerous single nucleotide polymorphisms (SNPs) have been reported to be associated with drug-resistance over the past decades - however, not all have sufficient evidence in the literature to support this association. The World Health Organisation (WHO) and others have graded reported drug-resistance SNPs into high, moderate and low confidence brackets ^{28,29}

### Targeted Next-Generation Sequencing

The WHO has announced a goal to effectively eradicate TB by 2035 and released guidelines on how to achieve that goal in 2015 ^{22,23,25,30}. Central to the WHO defined eradication strategy was a call for new diagnostic technologies and more rapid drug-susceptibility testing (DST) capabilities^{23,30-32}. Further was the requirement that these technologies should be effective for use in high-incidence, low-resource countries where the TB burden is high and medical infrastructure is generally lacking^{6,21,30}.

The non-molecular ‘gold-standard’ for detection of MTb and investigation of antibiotic resistance is culturing of a sample from a patient. However, culturing requires trained lab technicians and is typically extremely slow. The current ‘gold-standard’ molecular assay for detection of MTb and investigation of rifampicin (RIF) resistance (a surrogate marker for MDR-TB) is the Xpert MTB/RIF assay, a cartridge-based nucleic acid amplification test which can give rapid results. This test is easy to use, however, it can only identify RIF resistance so cannot diagnose XDR-TB ³³.

The FIND (Foundation for Innovative New Diagnostics) Seq&Treat programme (https://www.finddx.org/tb/seq-treat/) specifically called for the development of targeted next generation sequencing (tNGS) based tests for DR-TB that that could be evaluated by FIND and potentially endorsed by the WHO. Sequencing-based tests have the potential to detect all resistance associated SNPs, thereby determine which drugs will work best against the MTB strain infecting the patient (Kayomo et al. Sci Rep 10, 10786 (2020). https://doi.org/10.1038/s41598-020-67479-4).

tNGS allows sequencing of specific areas of the genome using next generation sequencing to detect variants within the regions of interest. There are different approaches to targeted sequencing, the most common being amplicon sequencing, which uses PCR primers to amplify the sequence/s of interest.

When multiple genes are to be targeted, multiplex polymerase chain reactions (multiplex PCRs) may be used to amplify several different DNA target sequences simultaneously. This process amplifies DNA in samples using multiple primers and a temperature-mediated DNA polymerase in a thermal cycler.

As drug-resistant SNPs are present at multiple sites across the genome, multiple regions need to be targeted by PCR. Multiplex PCR offers substantial advantages over amplification of single regions in separate reactions including higher throughput, cost savings (fewer deoxyribonucleotide triphosphates, enzymes, and other consumables required), turnaround time and production of more data from limited starting material.

Primer design for multiplexed PCR is, however, complex. The primers must have similar annealing temperatures, each pair needs to be specific for its target, and primer pairs should amplify similar sized PCR product to ensure similar amplification efficiency between the multiple targets in the reaction. In addition, as interaction between primers in multiplex reactions can reduce efficiency of amplification and the more primers in a reaction, the more likely this will occur. Designing efficient, sensitive and specific multiplex PCRs is challenging, and success is not assured.

Deeplex® Myc-TB, developed by Genoscreen, is an example of a targeted DR-TB test for prediction of resistance to 15 anti-tuberculous drugs, based on Illumina short read sequencing ^{34,35}(other tests have been developed but all have similar sensitivity and turnaround time). This test takes approximately 2 days to perform and has a limit of detection of ~1000 MTB cells. There remains a need for a more rapid and sensitive test.

It is an aim of the present invention to address the abovementioned problems and meet the abovementioned needs. Accordingly, it is an aim of the present invention to provide a method for rapidly and accurately detecting and/or identifying the presence of drug resistant mutations in a sample from subjects with suspected or confirmed TB using tNGS. It is a further aim to develop primers for achieving this objective, with a focus on the development of primers for use in multiplex PCRs. It is a further aim of the present invention to provide an assay or kit that addresses the abovementioned problems.

### SUMMARY

Single nucleotide polymorphisms (SNPs) known to confer resistance to first and second-line anti-TB drugs were selected, and primers developed for the selected targets and optimized for use in multiplex PCR. The gene targets were: eis, embB, rrs, rv0678, fabG1, gyrA, rpoB, ethA, rplC, katG, gidB, inha, rrl, pncA, rpsL, tlyA.

Accordingly, in a first aspect there is provided one or more oligonucleotide primer sets for amplifying a portion of one or more genes from M. tuberculosis and/or related bacteria in the M. tuberculosis complex selected from the group comprising or consisting of one or more of eis, embB, ethA, fabG1, gidB, gyrA, inha, katG, pncA, rrl, rplC, rpoB, rpsL, rrs, rv0678 and thyA, wherein each set comprises a pair of forward and reverse primers specific for said portion, wherein each primer has a sequence as set out in SEQ ID Nos. 1-33. Preferably, the one or more sets of primers are selected from SEQ ID Nos. 1-32.

In some embodiments, the oligonucleotide primer sets comprise or consist of one or more of SEQ ID Nos. 1 and 2; 3 and 4; 5 and 6; 7 and 8; 9 and 10; 11 and 12; 13 and 14; 15 and 16; 17 and 18; 19 and 20; 21 and 22; 23 and 24; 25 and 26; 27 and 28; 29 and 30; 31 and 32; and 19 and 33.

In some embodiments, the portion of the one or more genes contains one or more mutations, preferably one or more mutations that confer antibiotic resistance, preferably wherein the one or mutations are one or more single nucleotide polymorphisms that confer antibiotic resistance. In some embodiments, the antibiotic resistance is to one or more of ethambutol, isoniazid, pyrazinamide, rifampicin, streptomycin, amikacin, bedaquiline, capreomycin, ciprofloxacin, clofazimine, ethionamide, kanamycin, linezolid, moxifloxacin, ofloxacin and quinolones.

In some embodiments, the one or more genes are from the MTBc.

In some embodiments, the sets of oligonucleotide primers can be used for multiplex PCR. Sets of primers can thus be grouped into multiplex groups. In some embodiments, one or more multiplex groups can be formed. In some embodiments, multiplex groups can be formed each comprising one or more oligonucleotide primer sets as set out in SEQ ID Nos. 1-33, preferably SEQ ID Nos. 1-32. In some embodiments, one or more multiplex groups can be formed, each comprising oligonucleotide primer sets comprising or consisting of one or more of SEQ ID Nos. 1 and 2; 3 and 4; 5 and 6; 7 and 8; 9 and 10; 11 and 12; 13 and 14; 15 and 16; 17 and 18; 19 and 20; 21 and 22; 23 and 24; 25 and 26; 27 and 28; 29 and 30; 31 and 32; and 19 and 33.

In some embodiments, a multiplex group can comprise oligonucleotide primer sets for amplifying a portion of eis, embB, rrs, nv0678, and fabG1 (Group 1). In a further embodiment, a multiplex group can comprise oligonucleotide primer sets for amplifying a portion of gyrA, rpoB, ethA, rplC, and katG (Group 2). In a further embodiment, a multiplex group can comprise oligonucleotide primer sets for amplifying a portion of gidB, inhA, rrl, pncA, rpsL, and tlyA (Group 3). Accordingly, in some embodiments, groups of oligonucleotide primer sets comprise or consist of one or more of SEQ ID Nos. 1, 2, 3, 4, 5, 6, 7, 8, 9 and 10 (Group 1 in Table 7); one or more of SEQ ID Nos. 11, 12, 13, 14, 15, 16, 17, 18, 19 and 20 (Group 2 in Table 7); and/or one or more of SEQ ID Nos. 21, 22, 23, 24, 25, 26, 27, 28, 29, 30, 31 and 32 (Group 3 in Table 7).

Accordingly, in one embodiment there is provided one or more multiplex groups of oligonucleotide primer sets for amplifying a portion of genes from M. tuberculosis and/or related bacteria in the MTBc selected from the group comprising or consisting of one or more of eis, embB, ethA, fabG1, gidB, gyrA, inhA, katG, pncA,rrl, rplC, rpoB, rpsL, rrs, rv0678, tlyA, wherein each oligonucleotide primer set comprises or consists of a pair of forward and reverse primers specific for said portion, wherein the multiplex groups of oligonucleotide primer sets comprise or consist of one or more of SEQ ID Nos. 1, 2, 3, 4, 5, 6, 7, 8, 9 and 10 (Group 1 in Table 7); one or more of SEQ ID Nos. 11, 12, 13, 14, 15, 16, 17, 18, 19 and 20 (Group 2 in Table 7); and/or one or more of SEQ ID Nos. 21, 22, 23, 24, 25, 26, 27, 28, 29, 30, 31 and 32 (Group 3 in Table 7).

In some such embodiments, the multiplex groups of oligonucleotide primer sets comprise or consist of one or more of SEQ ID Nos. 1 and 2; 3 and 4; 5 and 6; 7 and 8; and 9 and 10 (Group 1 in Table 7); one or more of SEQ ID Nos. 11 and 12; 13 and 14; 15 and 16; 17 and 18; and 19 and 20 (Group 2 in Table 7); and/or one or more of SEQ ID Nos. 21 and 22; 23 and 24; 25 and 26; 27 and 28; 29 and 30; and 31 and 32 (Group 3 in Table 7). In some embodiments, a multiplex group of oligonucleotide primer sets comprises or consists of one or more of SEQ ID Nos. 1 and 2; 3 and 4; 5 and 6; 7 and 8; and 9 and 10 (Group 1 in Table 7). In some embodiments, a multiplex group of oligonucleotide primer sets comprises or consists of one or more of SEQ ID Nos. 11 and 12; 13 and 14; 15 and 16; 17 and 18; and 19 and 20 (Group 2 in Table 7). In some embodiments, a multiplex group of oligonucleotide primer sets comprises or consists of one or more of SEQ ID Nos. 21 and 22; 23 and 24; 25 and 26; 27 and 28; 29 and 30; and 31 and 32 (Group 3 in Table 7).

In a second aspect there is provided a multiplex PCR reaction mixture comprising one or more groups of oligonucleotide primer sets for amplifying a portion of one or more genes from M. tuberculosis and/or related bacteria in the M. tuberculosis complex selected from the group comprising or consisting of one or more of eis, embB,ethA, fabG1, gidB, gyrA, inha, katG, pncA, rrl, rplC, rpoB,rpsL, rrs, rv0678, tlyA, wherein each set comprises a pair of forward and reverse primers specific for said portion, wherein the groups of oligonucleotide primer sets comprise or consist of one or more of SEQ ID Nos. 1, 2, 3, 4, 5, 6, 7, 8, 9 and 10 (Group 1 in Table 7); one or more of SEQ ID Nos. 11, 12, 13, 14, 15, 16, 17, 18, 19 and 20 (Group 2 in Table 7); and/or one or more of SEQ ID Nos. 21, 22, 23, 24, 25, 26, 27, 28, 29, 30, 31 and 32 (Group 3 in Table 7).

In some embodiments, a multiplex PCR reaction mixture comprises a group of oligonucleotide primer sets comprising or consisting of one or more of SEQ ID Nos. 1 and 2; 3 and 4; 5 and 6; 7 and 8; and 9 and 10 (Group 1 in Table 7); one or more of SEQ ID Nos. 11 and 12; 13 and 14; 15 and 16; 17 and 18; and 19 and 20 (Group 2 in Table 7); and/or one or more of SEQ ID Nos. 21 and 22; 23 and 24; 25 and 26; 27 and 28; 29 and 30; and 31 and 32 (Group 3 in Table 7). In one embodiment, a multiplex PCR reaction mixture comprises a group of oligonucleotide primer sets comprising or consisting of SEQ ID Nos. 1 and 2; 3 and 4; 5 and 6; 7 and 8; and 9 and 10 (Group 1 in Table 7). In a further embodiment, a multiplex PCR reaction mixture comprises a group of oligonucleotide primer sets comprising or consisting of one or more of SEQ ID Nos. 11 and 12; 13 and 14; 15 and 16; 17 and 18; and 19 and 20 (Group 2 in Table 7). In a further embodiment, a multiplex PCR reaction mixture comprises a group of oligonucleotide primer sets comprising or consisting of SEQ ID Nos. 21 and 22; 23 and 24; 25 and 26; 27 and 28; 29 and 30; and 31 and 32 (Group 3 in Table 7).

The multiplex PCR reaction mixture may comprise further ingredients and reagents required to perform multiplex PCR, such as buffers, deoxynucleotide triphosphates (dNTPs), DMSO, water and DNA polymerase.

In some multiplex embodiments, said primers may be mixed to a working concentration of 0.2 µM. Further typically with the exception of tlyA which requires a working concentration of 0.3 µM, for consistent target amplification.

In some embodiments, the portion of the one or more genes from M. tuberculosis and/or related bacteria in the M. tuberculosis complex is obtained from a sample from a subject suspected or confirmed to have TB. The sample may be one or more tissues and/or bodily fluids obtained from the subject, including one or more of sputum; urine; blood; plasma; serum; synovial fluid; pus; cerebrospinal fluid; pleural fluid; pericardial fluid; ascitic fluid; sweat; saliva; tears; vaginal fluid; semen; interstitial fluid; bronchoalveolar lavage; bronchial wash; gastric lavage; gastric wash; a transtracheal or transbronchial fine needle aspiration; bone marrow; pleural tissue; tissue from a lymph node, mediastinoscopy, thoracoscopy or transbronchial biopsy; or combinations thereof; or a culture specimen of one or more tissues and/or bodily fluids obtained from a subject suspected of having or confirmed to have TB. Typically, the sample includes cells and/or DNA from M. tuberculosis and/or related bacteria in the M. tuberculosis complex.

In a third aspect there is provided a method of detecting and/or identifying the presence of one or more mutations that confer antibiotic resistance in a sample comprising DNA from Mycobacterium tuberculosis and/or related bacteria in the M. tuberculosis complex, said method including the steps of;
(a) isolating or extracting DNA from the sample;
(b) amplifying relevant gene regions or amplicons by multiplex polymerase chain reaction using one or more groups of oligonucleotide primer sets according to the first aspect;
(c) subjecting the amplified gene regions or amplicons to DNA sequencing; and
detecting one or more mutations.

In some embodiments, the mutations are within one or more genes selected from the group consisting of one or more of eis, embB, ethA, fabG1, gidB, gyrA, inha, katG, pncA, rrl, rplC, rpoB, rpsL, rrs, rv0678 and tlyA.

In some embodiments the mutations are one or more single nucleotide polymorphisms.

In some embodiments, the antibiotic resistance is to one or more of ethambutol, isoniazid, pyrazinamide, rifampicin, streptomycin, amikacin, bedaquiline, capreomycin, ciprofloxacin, clofazimine, ethionamide, kanamycin, linezolid, moxifloxacin, ofloxacin and quinolones.

The amplification step uses one or more groups of oligonucleotide primer sets. In some embodiments, the groups of oligonucleotide primer sets comprise or consist of one or more forward and reverse primer pairs selected from SEQ ID Nos. 1 and 2; 3 and 4; 5 and 6; 7 and 8; 9 and 10; 11 and 12; 13 and 14; 15 and 16; 17 and 18; 19 and 20; 21 and 22; 23 and 24; 25 and 26; 27 and 28; 29 and 30; 31 and 32 and 19 and 33.

In some embodiments, the one or more groups of oligonucleotide primer sets comprise or consist of one or more of SEQ ID Nos. 1, 2, 3, 4, 5, 6, 7, 8, 9 and 10 (Group 1 in Table 7); one or more of SEQ ID Nos. 11, 12, 13, 14, 15, 16, 17, 18, 19 and 20 (Group 2 in Table 7) and/or one or more of SEQ ID Nos. 21, 22, 23, 24, 25, 26, 27, 28, 29, 30, 31 and 32 (Group 3 in Table 7). In some embodiments, the amplification step uses a group of oligonucleotide primer sets consisting of SEQ ID Nos. 1, 2, 3, 4, 5, 6, 7, 8, 9 and 10 (Group 1). In some embodiments, the amplification step uses a group of oligonucleotide primer sets consisting of SEQ ID Nos. 11, 12, 13, 14, 15, 16, 17, 18, 19 and 20 (Group 2 in Table 7). In some embodiments the amplification step uses a group of oligonucleotide primer sets consisting of SEQ ID Nos. 21, 22, 23, 24, 25, 26, 27, 28, 29, 30, 31 and 32 (Group 3 in Table 7).

Detection of a mutation is indicative of antibiotic resistance. Identification of the mutation informs or allows identification of the nature of the antibiotic resistance (i.e. the antibiotic to which the bacteria is resistant).

Accordingly, in a fourth aspect there is provided a method of predicting whether a patient suffering from tuberculosis will respond to treatment with one or more of ethambutol, isoniazid, pyrazinamide, rifampicin, streptomycin, amikacin, bedaquiline, capreomycin, ciprofloxacin, clofazimine, ethionamide, kanamycin, linezolid and moxifloxacin, said method comprising a step of determining the presence of one or more drug resistant mutations in one or more genes selected from the group comprising one or more of eis, embB, ethA, f fabG1, gidB, gyrA, inha, katG, pncA, rrl, rplC, rpoB, rpsL, rrs, rv0678 and tlyA in DNA obtained from a sample from the patient, the method comprising:
(a) isolating or extracting DNA from the sample;
(b) amplifying relevant gene regions or amplicons by multiplex polymerase chain reaction using one or more groups of oligonucleotide primer according to the first aspect;
(c) subjecting the amplified gene regions or amplicons to DNA sequencing; and
detecting the one or more mutations.

In some embodiments, the method is for predicting whether a patient suffering from tuberculosis will respond to treatment with one or more of ethambutol, isoniazid, streptomycin, amikacin, bedaquiline, capreomycin, clofazimine, ethionamide, kanamycin, wherein the one or more genes are eis, embB, rrs, rv0678, and fabG1; and the group of oligonucleotide primer sets consists of SEQ ID Nos. 1, 2, 3, 4, 5, 6, 7, 8, 9 and 10 (Group 1 in Table 7).

In some embodiments, the method is for predicting whether a patient suffering from tuberculosis will respond to treatment with one or more of isoniazid, rifampicin, ciprofloxacin, ethionamide, linezolid, moxifloxacin, ofloxacin and quinolones, wherein the one or more genes are gyrA, rpoB, ethA, rplC, and katG; and the group of oligonucleotide primer sets consists of SEQ ID Nos. 11, 12, 13, 14, 15, 16, 17, 18, 19 and 20 (Group 2 in Table 7).

In some embodiments, the method is for predicting whether a patient suffering from tuberculosis will respond to treatment with one or more of pyrazinamide, streptomycin, capreomycin and ethionamide, wherein the one or more genes are gidB, inha, rrl, pncA, rpsL, and tlyA; and the group of oligonucleotide primer sets consists of SEQ ID Nos. 21, 22, 23, 24, 25, 26, 27, 28, 29, 30, 31 and 32 (Group 3 in Table 7).

In some embodiments according to the third or fourth aspect, the DNA is from M. tuberculosis.

In some embodiments according to the third or fourth aspect, the sample is a clinical sample. The sample may be one or more tissues and/or bodily fluids obtained from a subjected suspected of having or confirmed to have TB, including one or more of sputum; urine; blood; plasma; serum; synovial fluid; pus; cerebrospinal fluid; pleural fluid; pericardial fluid; ascitic fluid; sweat; saliva; tears; vaginal fluid; semen; interstitial fluid; bronchoalveolar lavage; bronchial wash; gastric lavage; gastric wash; a transtracheal or transbronchial fine needle aspiration; bone marrow; pleural tissue; tissue from a lymph node, mediastinoscopy, thoracoscopy or transbronchial biopsy; or combinations thereof; or a culture specimen of one or more tissues and/or bodily fluids obtained from a subject suspected of having or confirmed to have TB. Typically, the sample includes cells and/or DNA from M. tuberculosis and/or related bacteria in the M. tuberculosis complex. In some embodiments, the sample is a sputum sample from a subject suspected or confirmed to have TB.

In some embodiments, the samples undergo mechanical disruption in order to disrupt the cells in the sample and achieve cell lysis. Any suitable means may be used, for example bead beating.

The step of isolating or extracting DNA from the sample may be carried out by any suitable means, including by the use of an appropriate kit, using given or standard protocols. For example, a Maxwell RSC PureFood Pathogen Kit from Promega AS1660, with instructions for use. In some embodiments, a Maxwell RSC PureFood Pathogen Kit from Promega AS1660 may be used. In some such embodiments, the following modifications were made from the kit instructions: The kit teaches use of a 800 µl sample; in some embodiments, a 400 µl sample after bead beating was used. The kit teaches adding 200 µl lysis buffer A and incubating at 56° C. for 4 min with shaking; in some embodiments, 200 µl lysis buffer A was added together with 40 µl Proteinase k, with incubation at 65° C. for 10 min. The kit teaches addition of 300 µl of lysis buffer and then placing the sample on the robot; in some embodiments, 300 µl lysis buffer was added together with 400 µl PBS and the sample was then placed on the robot.

In embodiments according to the third or fourth aspect wherein more than one group of primer sets are used for the amplification step, each group may be run as a separate multiplex group template.

Labelled nucleotides or labelled primers may be used in the amplification of the DNA for the purpose of, for example, quality control. For example, a fluorescent DNA-binding dye may be added to enable DNA quantitation. Any suitable dyes or probes with dyes may be used, such as probes with fluorescent dyes, such as use of a sybr green assay such as Roche Lightcycler® 480 SYBR Green I master.

In embodiments wherein more than one group of primer sets are used for the amplification step and each group is run as a separate multiplex group template, one or more multiplex group templates may be pooled to make a single template for DNA quantitation and/or sequencing.

Samples may then undergo barcode ligation and adaptor ligation to create a library for sequencing. Barcoding can be used when the amount of data required per sample is less than the total amount of data that can be generated: it allows pooling of multiple samples and sequencing of them together. Any suitable means may be used, including the use of barcoding kits, using given or standard protocols. For example, Oxford Nanopore Technologies provides amplicon barcoding with native barcoding expansion 96 (EXP-NBD196 and SQK-LSK109), including instructions for use. In some embodiments, the Oxford Nanopore Technologies amplicon barcoding with native barcoding expansion 96 (EXP-NBD196 and SQK-LSK109) may be used following the instructions for use provided.

The DNA sequencing step may be carried out by any suitable means. In preferred embodiments, the DNA sequencing is tNGS or third-generation sequencing (also known as long-read sequencing). Third-generation sequencing may be carried out using Oxford Nanopore Technologies’ MinION, or PacBio’s sequencing platform of single molecule real time sequencing (SMRT). Oxford Nanopore’s sequencing technology is based on detecting the changes in electrical current passing through a nanopore as a piece of DNA moves through the pore. The current measurably changes as the bases G, A, T and C pass through the pore in different combinations. SMRT is based on the properties of zero-mode waveguides. Signals in the form of fluorescent light emission from each nucleotide are incorporated by a DNA polymerase bound to the bottom of the zL well. In preferred embodiments the sequencing is long-read nanopore sequencing.

The step of detecting of one or more mutations may be carried out by any suitable method, such as suitable bioinformatics tools and programmes. In some embodiments, the Oxford nanopore technologies workflow for TB may be used in desktop program EPI2ME with the FASTQ TB RESISTANCE PROFILE v2020.03.11.

The oligonucleotide primer sets of the first aspect, the PCR reaction mixture of the second aspect and/or the method of the third aspect can be used to identify both the presence and identity of drug resistance mutations in the genes of TB bacteria from a particular subject. Such information informs decisions regarding drug administration and allows a tailored treatment regime to be determined for the patient depending upon the identified mutations.

As such, in a fifth aspect, there is provided a method for determining an appropriate antibiotic treatment regime for a patient with tuberculosis, comprising detecting and/or identifying the presence of one or more mutations that confer antibiotic resistance in a sample from the patient according to the third aspect, and determining an appropriate antibiotic regime on the basis of the mutations detected/identified. The disclosure herein also provides a method of assigning a patient with tuberculosis to one of a certain number of treatment pathways comprising detecting and/or identifying the presence of one or more mutations that confer antibiotic resistance in a sample from the patient using a method according to the third aspect, and assigning the patient to a treatment regime on the basis of the mutations detected/identified.

In a sixth aspect there is provided a kit comprising one or more oligonucleotide primer sets or groups of oligonucleotide primer sets according to the first aspect. The kit may be used to carry out a method according to one or more of steps (a) (b) or (c) of the third aspect. The kit may further comprise ingredients and reagents required to carry out the method according to one or more of steps (a) (b) or (c) of the third aspect, including buffers, DNA polymerase and nucleotides. In some embodiments, the kit further comprises reagents required for the amplification of the gene regions between the primers. The kit may further comprise a sample collection container for receiving the sample. Samples may be processed according to the method of the third aspect immediately, alternatively they may be stored at low temperatures, for example in a fridge or freezer before the method is carried out. The sample may be processed before the method is carried out. For instance, a sedimentation assay may be carried out, and/or a preservative and/or dilutant may be added. Thus, the sample collection container may contain suitable processing solutions, such as buffers, preservative and dilutants.

Gene targets and their corresponding primer pairs according to the disclosure herein are as shown in Table 1.

TABLE 1

| | | |
|---|---|---|
| Gene Target | Forward Primer (5′-3′) | Reverse Primer (5′-3′) |
| eis | TGTCGGGTACCTTTCGAGC SEQ ID. No. 1 | TCCATGTACAGCGCCATCC SEQ ID. No. 2 |
| embB | CGCCGTGGTGATATTCGGC SEQ ID. No. 3 | GCACACCGTAGCTGGAGAC SEQ ID. No. 4 |
| rrs | CTCTGGGCAGTAACTGACGC SEQ ID. No. 5 | GAGTGTTGCCTCAGGACCC SEQ ID. No. 6 |
| rv0678 | GCTCGTCCTTCACTTCGCC SEQ ID. No. 7 | ATCAGTCGTCCTCTCCGGT SEQ ID. No. 8 |
| fabG1 | CTTTTGCACGCAATTGCGC SEQ ID. No. 9 | AGCAGTCCTGTCATGTGCG SEQ ID. No. 10 |
| gyrA | TGACAGACACGACGTTGCC SEQ ID. No. 11 | CGATCGCTAGCATGTTGGC SEQ ID. No. 12 |
| rpoB | TCATCATCAACGGGACCGAG SEQ ID. No. 13 | ACACGATCTCGTCGCTAACC SEQ ID. No. 14 |
| ethA | TGGATCCATGACCGAGCAC SEQ ID. No. 15 | GTCCAGGAGGCATTGGTGT SEQ ID. No. 16 |
| rplC | AGTACAAGGACTCGCGGGA SEQ ID. No. 17 | TCGAGTGGGTACCCTGGC SEQ ID. No. 18 |
| katG redesigned | CTGTGGCCGGTCAAGAAGA | GGATCTGGCTCTTAAGGCTGG |
| | SEQ ID. No. 19 | SEQ ID. No. 20 |
| gidb | TGACACAGACCTCACGAGC SEQ ID. No. 21 | GCCCTTCTGATTCGCGATG SEQ ID. No. 22 |
| inhA | GGGCGCTGCAATTTATCCC SEQ ID. No. 23 | GGCGTAGATGATGTCACCC SEQ ID. No. 24 |
| rrl | GGTCCGTGCGAAGTCGC SEQ ID. No. 25 | TGAACCCGTGTTCTGCGG SEQ ID. No. 26 |
| pncA | TCACCGGACGGATTTGTCG SEQ ID. No. 27 | TCCAGATCGCGATGGAACG SEQ ID. No. 28 |
| rpsL | GCGGCGGGTATTGTGGTT SEQ ID. No. 29 | TAACCGGCGCTTCTCACC SEQ ID. No. 30 |
| tlyA | CGTTGATGCGCAGCGATC SEQ ID. No. 31 | GGTCTCGGTGGCTTCGTC SEQ ID. No. 32 |
| katG initial | CTGTGGCCGGTCAAGAAGA SEQ ID. No. 19 | TGCCCGGATCTGGCTCTTA SEQ ID. No. 33 |

### BRIEF DESCRIPTION OF FIGURES

For a better understanding of the invention, and to show how embodiments of the same may be carried into effect, reference will now be made, by way of example, to the accompanying Figures in which:

FIG. 1: qPCR curves showing nested qPCR amplification of multiplexed primers;

FIG. 2: Fragment size analysis of amplicons produced during each triplex reaction. A1- ladder, B1 - triplex 1, C1 - triplex 2, D1 - triplex 3, E1 - triplex 4 and F1 - triplex 5;

FIG. 3: Example of nested qPCR results testing the amplification efficiency of individual gene targets within multiplex version 4, group 1;

FIG. 4: TapeStation imaging of 5-plex PCR products;

FIG. 5: Nested qPCR results for gene targets in multiplex group formulation 7;

FIG. 6: Nested qPCR results for gene targets in Multiplex group formulation 9, Group 2.

### DETAILED DESCRIPTION

### Detectable Drug-Resistance SNPs

Selected target single nucleotide polymorphisms (SNPs) that confer resistance to first and second-line anti-TB drugs were chosen primarily from WHO/FIND evidence published in the WHO next-generation sequencing technical guide ³⁶. The targets for rpsL were selected from prior literature by Karimi, et al. and Meier, et al^{37,38}. Targets for gidB were selected on evidence from Villellas, et al³⁹. Targets for ethA were selected on evidence from Morlock, et al⁴⁰. Targets for embB were selected on evidence from Zhao, et al⁴¹. Finally, targets for tlyA were selected from prior literature by Maus, et al⁴².

Base positions and genes as listed are based on the H37Rv M. tuberculosis reference genome available through the NCBI database (NC_000962.3)⁴³. Targeted mutations were identified either as their codon location or their nucleotide location. Mutations were identified by the codon which they effect when the SNP occurs within an annotated gene region and the prior literature explicitly states the altered amino acid. Targets were listed by nucleotide mutation in the event they occur within a gene promoter region or the supporting literature does not explicitly identify the amino acid mutation. These promoter region SNPs are further identified by a “-” prior to its position indicating it occurs before the annotated gene. The effect of the mutated base is also included; e.g. Asparagine to Histidine or nucleotide A to nucleotide C (Table A, appended).

### Multiplex Group Optimisation

Primers were developed for the chosen gene/promotor targets (n=16; Table 2) that amplified ~1000 bp regions containing the targeted SNPs of interest. As discussed above, interaction between primers in multiplex reactions can reduce efficiency of amplification and the more primers in a reaction, the more likely this will occur. Therefore designing efficient, sensitive and specific multiplex PCRs is complex.

TABLE 2

Details of genes conferring drug resistance

| | |
|---|---|
| Drug | Genes conferring resistance |
| Ethambutol | embB |
| Isoniazid | fabG1 |
| | inhA |
| | katG |
| Pyrazinamide | pncA |
| Rifampicin | rpoB |
| Streptomycin | gidB |
| | rpsL |
| | rrs |
| Amikacin | rrs |
| Bedaquiline | rv0678 |
| Capreomycin | gidB |
| | rrs |
| | tlyA |
| Ciprofloxacin | gyrA |
| Clofazimine | rv0678 |
| Ethionamide | ethA |
| | fabG1 |
| | inhA |
| Kanamycin | eis |
| | rrs |
| Linezolid | rplC |
| Moxifloxacin | gyrA |
| Ofloxacin | gyrA |
| Quinolones | gyrA |

The following genes were targeted in the DR-TB sequencing assay: eis, embB, ethA, fabG1, gidB, gyrA, inha, katG, pncA, rrl, rplC, rpoB, rpsL, rrs, rv0678, tlyA. Initially, gene target primer pairs were grouped into 5 sets of three (Table 3). DNA was extracted from M. bovis BCG and used to test the specificity and sensitivity of the triplex assays.

TABLE 3

Gene targets per triplex

| | | | |
|---|---|---|---|
| | Gene Target 1 | Gene Target 2 | Gene Target 3 |
| Triplex 1 | Eis | ethA | embB |
| Triplex 2 | pncA | gyrA | rpoB |
| Triplex 3 | fabG1/inhA | rrs | gidB |
| Triplex 4 | rv0678 | rplC | katG |
| Triplex 5 | tlyA | rpsL | rrl |

The multiplex PCRs were performed as follows:
Per reaction:
5 µl DNA (concentration approx. 20 ng)
25 µl Qiagen 2x Multiplex Master Mix
10 µL Qiagen 5x Q-Solution
2.5 µl (10 µM, final conc 0.2 µM) Forward Multiplex Primer
2.5 µl (10 µM, final conc 0.2 µM) Reverse Multiplex Primer
5 µl Molecular H₂O

PCR conditions:
Cycling Conditions

| Step | Temperature (C) | Time (mm:ss) | # Cycles |
|---|---|---|---|
| Pre-Incubation Pre-Incubation | 95 | 20:00 | 1 |
| Amplification | 9 4 | 94 00:30 | 35 |
| | 60 | 01:30 | |
| | 72 | 1:30 | |
| Extension | 72 | 10:00 | 1 1 |
| Hold | 4 | ∞ | 1 |

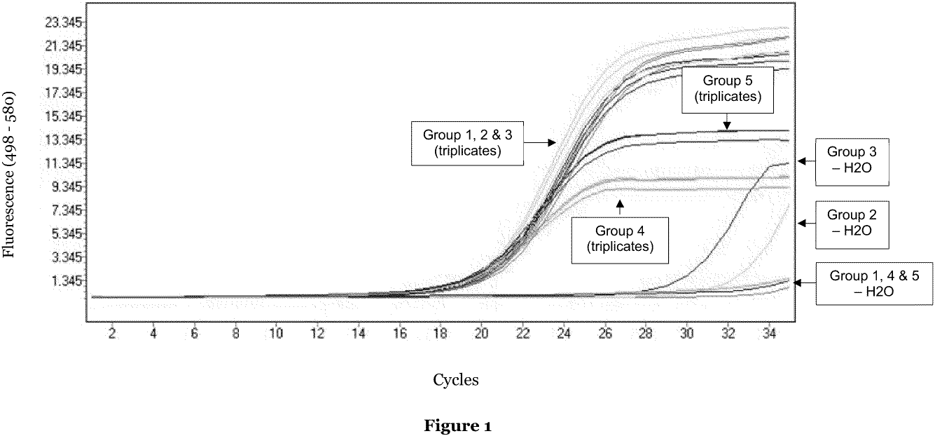

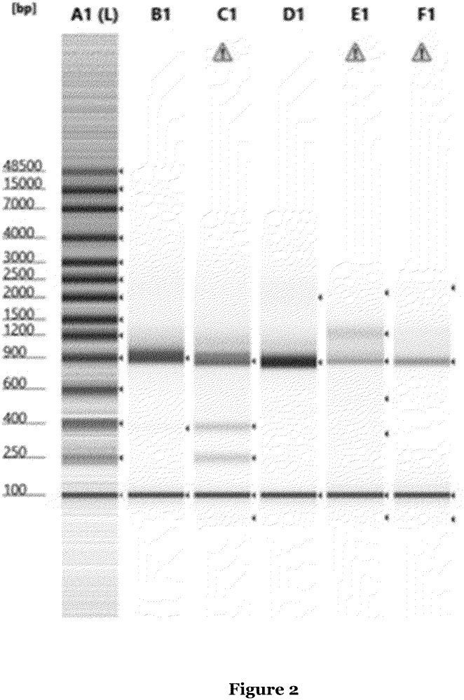

Nested qPCR was performed on the amplified products from the multiplex PCR to evaluate the amplification of all the targets. Nested PCR on all amplified products resulted in very similar Ct values, indicating the same amplification efficiency across all primers (FIG. 1). Fragment size analysis of the multiplex PCR amplicons expected at ~1000bp showed minimal non-specific amplification with additional amplicon bands only seen in Triplex 2 and Triplex 5 (FIG. 2: A1 -ladder, B1 - triplex 1, C1 - triplex 2, D1 - triplex 3, E1 - triplex 4 and F1 - triplex 5).

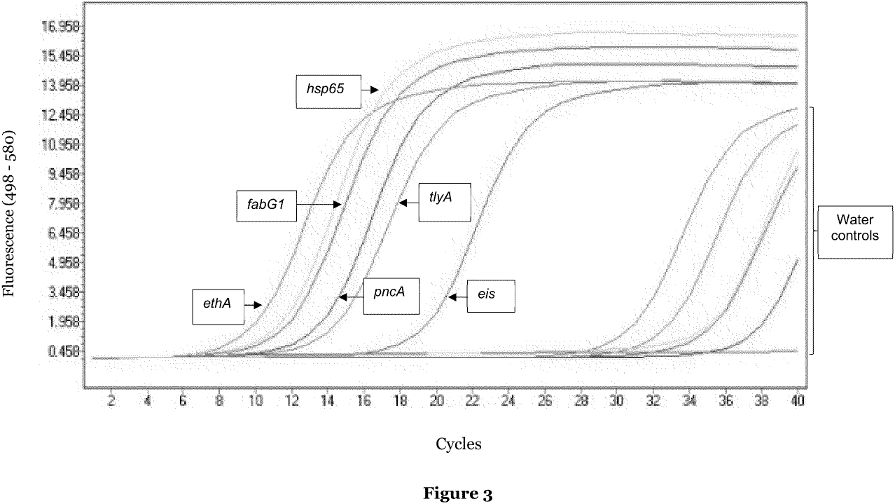

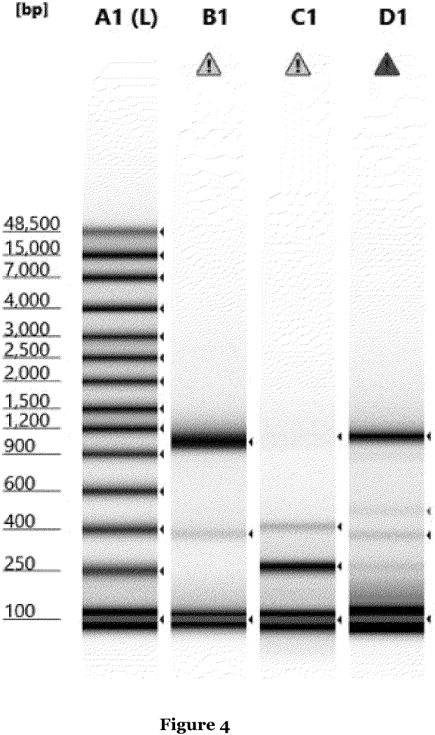

While the triplex assays worked well, the requirement for 5 PCR reactions was considered too laborious and expensive for the tNGS assay. Hence, the primer pairs were combined in a new format to make three groups (two 5-plex and one 6-plex reaction), in order to simplify the assay. Multiplex efficiency was again measured by nested qPCR (FIG. 3: Ct values range from 8-18 indicating inefficient amplification of some targets caused by primer interaction) and fragment size analysis was used to show any non-specific amplification (FIG. 4: Results show non-specific amplification in Group 2 (C1) with no visible band of expected size (~1000 bp). Group 1 and Group 3 show less non-specific amplification but qPCR results showed inefficient amplification of some targets). Multiple multiplex primer combinations had to be tested as primer interaction led to amplification inefficiencies of one or more targets per multiplex. In total, nine different combinations were tested (Table 4). A new target for identifying Mycobacterium species, hsp65, was introduced at version 3. This was designed to provide more information in a case where a sample is negative for MTBC.

TABLE 4

The versions of the multiplex formulations tested during the optimisation process

| Multiplex Design Group Formulation Version | Group 1 Gene Targets | Group 2 Gene Targets | Group 3 Gene Targets |
|---|---|---|---|
| 1 | eis, ethA, embB, tlyA, rv0678 | pncA, gyrA, rpoB, rpsL, rplC | fabG1, inha, rrs, gidB, rrl, katG |
| 2 | eis, ethA embB, tlyA, pncA. | gyrA, rpoB, rpsL, rplC, rv0678 | fabG1, inha, rrs, gidB, rrl, katG |
| 3 | eis, embB, ethA, pncA, tlyA, hsp65 | gyrA, rpoB, fabG1, rpsL, rplC, rv0678 | inha, rrs, gidB, rrl, katG |
| 4 | eis, ethA, pncA, tlyA, hsp65, fabG1 | gyrA, rpoB, rpsL, rplC, rv0678, embB | inha, rrs, gidB, rrl, katG |
| 5 | ethA, pncA, hsp65, rrs, embB | gyrA, rpoB, rpsL, rplC, rv0678, fabG1 | inha, gidB, rrl, katG, eis, tlyA |
| 6 | hsp65, rrs, rpsL, fabG1, tlyA | gyrA, rpoB, rplC, rv0678, ethA, embB | inha, gidB, rrl, katG, eis, pncA |
| 7 | fabG1, rrs, rv0678, eis, embB | gyrA, rpoB, rplC, ethA, katG, hsp65 | inhA, gidB, rrl, pncA, rpsL, tlyA |
| 8 | fabG1, rrs, rv0678, ethA, inhA | gyrA, rpoB, rplC, katG, hsp65, embB | gidB, rrl, pncA, rpsL, tlyA, eis |
| 9 | fabG1, rrs, rv0678, ethA, inhA | gyrA, rpoB, rplC, katG, embB | gidB, rrl, pncA, rpsL, tlyA, eis |

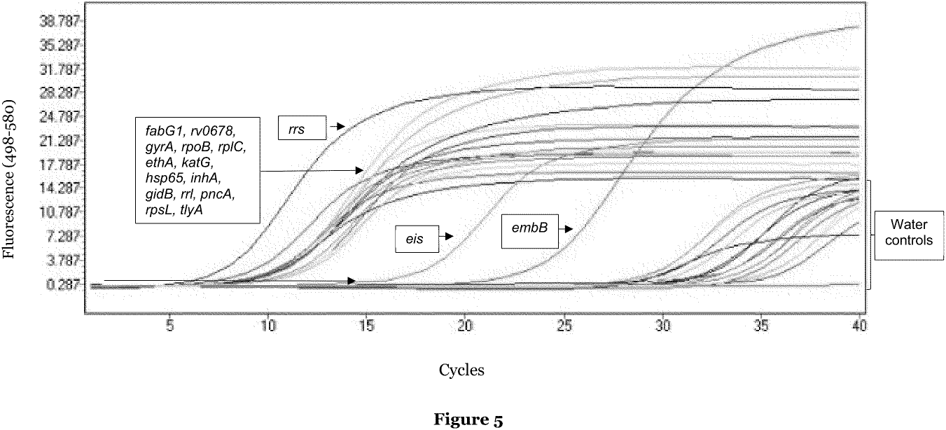

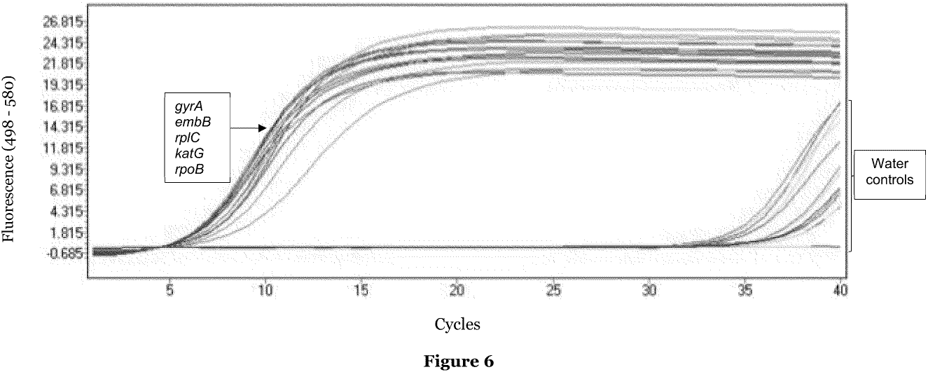

Formulations 1-6 had multiple late Cts and/or total dropouts indicative of inhibition and competition within the multiplex groups. Version 7 showed multiplex groups 2 and 3 had Ct ranges <1.5 while group 1 had a range of approximately 15Cts (FIG. 5). Subsequent optimisations led to two more versions, resulting in the final version 9 which had all multiplex group Ct ranges <2 (FIG. 6).

### Final Primer Design

Concurrently to optimising the group formulations, various primers were redesigned to overcome primer interactions. In total there were 48 multiplex primer combinations with >300 primer designs (Table 5) before the optimal sequences were determined.

After testing ~400 samples provided by FIND in a lab validation study (described below), a redesign was required for the katG reverse primer to avoid a common non-resistance conferring SNP in the primer binding site. To overcome this, five new reverse primers were tested where each primer was shifted towards the 3′ 1 bp at a time (up to 5bp shift) (Table 6). Option 5 was selected for the final assay as the mutation site was avoided and the performance of the assay wasn’t negatively affected.

TABLE 6

Redesigned katG primer options (non-resistance conferring SNP in bold).

| Base Pair Positions Shifted Toward 3′ End | Primer sequence (5′-3′) |
|---|---|
| Original Primer | TGCCCGGATCTGGCTCTTA |
| 1 | GCCCGGATCTGGCTCTTAA |
| 2 | CCCGGATCTGGCTCTTAAGG |
| 3 | CCGGATCTGGCTCTTAAGGC |
| 4 | CGGATCTGGCTCTTAAGGCTG |
| 5 | GGATCTGGCTCTTAAGGCTGG |

The final optimal iteration of primers consisted of two 5-plex groups and one 6-plex group (Table 7).

TABLE 7

Primer sequences

| | | | |
|---|---|---|---|
| Multiplex Group | Target and Orientation | Sequence (5′ - 3′) | SEQ ID No |
| Group 1 | eis Forward | TGTCGGGTACCTTTCGAGC | SEQ ID No.1 |
| | eis Reverse | TCCATGTACAGCGCCATCC | SEQ ID No.2 |
| | embB Forward | CGCCGTGGTGATATTCGGC | SEQ ID No.3 |
| | embB Reverse | GCACACCGTAGCTGGAGAC | SEQ ID No.4 |
| | rrs Forward | CTCTGGGCAGTAACTGACGC | SEQ ID No.5 |
| | rrs Reverse | GAGTGTTGCCTCAGGACCC | SEQ ID No.6 |
| | rv0678 Forward | GCTCGTCCTTCACTTCGCC | SEQ ID No.7 |
| | rv0678 Reverse | ATCAGTCGTCCTCTCCGGT | SEQ ID No.8 |
| | fabG1 Forward | CTTTTGCACGCAATTGCGC | SEQ ID No.9 |
| | fabG1 Reverse | AGCAGTCCTGTCATGTGCG | SEQ ID No.10 |
| Group 2 | gyrA Forward | TGACAGACACGACGTTGCC | SEQ ID No.11 |
| | gyrA Reverse | CGATCGCTAGCATGTTGGC | SEQ ID No.12 |
| | rpoB Forward | TCATCATCAACGGGACCGAG | SEQ ID No.13 |
| | rpoB Reverse | ACACGATCTCGTCGCTAACC | SEQ ID No.14 |
| | ethA Forward | TGGATCCATGACCGAGCAC | SEQ ID No.15 |
| | ethA Reverse | GTCCAGGAGGCATTGGTGT | SEQ ID No.16 |
| | rplC Forward | AGTACAAGGACTCGCGGGA | SEQ ID No.17 |
| | rplC Reverse | TCGAGTGGGTACCCTGGC | SEQ ID No.18 |
| | katG Forward | CTGTGGCCGGTCAAGAAGA | SEQ ID No.19 |
| | katG Reverse redesigned | GGATCTGGCTCTTAAGGCTGG | SEQ ID No.20 |
| Group 3 | gidB Forward | TGACACAGACCTCACGAGC | SEQ ID No.21 |
| | gidB Reverse | GCCCTTCTGATTCGCGATG | SEQ ID No.22 |
| | inhA Forward | GGGCGCTGCAATTTATCCC | SEQ ID No.23 |
| | inhA Reverse | GGCGTAGATGATGTCACCC | SEQ ID No.24 |
| | rrl Forward | GGTCCGTGCGAAGTCGC | SEQ ID No.25 |
| | rrl Reverse | TGAACCCGTGTTCTGCGG | SEQ ID No.26 |
| | pncA Forward | TCACCGGACGGATTTGTCG | SEQ ID No.27 |
| | pncA Reverse | TCCAGATCGCGATGGAACG | SEQ ID No.28 |
| | rpsL Forward | GCGGCGGGTATTGTGGTT | SEQ ID No.29 |
| | rpsL Reverse | TAACCGGCGCTTCTCACC | SEQ ID No.30 |
| | tlyA Forward | CGTTGATGCGCAGCGATC | SEQ ID No.31 |
| | tlyA Reverse | GGTCTCGGTGGCTTCGTC | SEQ ID No.32 |

### Gene Target Regions

Visualized target regions are shown as either the parent or complement strand depending on gene orientation. Target regions were designed to be 900-1100 bp long as this is a good size for PCR and nanopore sequencing. Keeping the PCR products a uniform size reduces bias toward certain targets in multiplex PCR and sequencing reactions.

### Eis

The target region for identified eis mutations encompasses the promoter region, denoted in bold text, of the 1,209 base pair eis gene. The eis gene is on the complement strand. Sequence outside the annotated gene is highlighted in grey. Forward and reverse primer locations are written in italics.

Forward Primer: 5′-TGTCGGGTACCTTTCGAGC-3′ [Sequence ID No. 1]

Reverse Primer: 5′-TCCATGTACAGCGCCATCC-3′ [Sequence ID No. 2]

TGTCGGGTACCTTTCGAGCCGCCGAGCTGACCGCGGCGGAACTAGGGTCC
CGCCGTTAGGGTGATCGACTCGAGGTCGGCCGCGATGGGCGTCGGTTTCG
CGGCACTGGCGGCCGGGCGGGCAGCCGCCGCAGGCGATGAGCTGGATACG
TCGCGCGCGCAGCGGCTGCGGCGGTAAGCCGGATTCACGCGTTCGTCGCT
GTAGCGCGGTTGGACAATCTGCGCCGCAGCGGGCGCATCAGTGGGGCCAA
GGCATGGTTGGGCACCGCGCTGGCGCTCAAGCCGCTGCTGTCAGTCGACG
ACGGAAAACTTGTTCTGGTCCAACGGGTTCGCACTGTGAGCAACGCGACG
GCGGTGATGATCGACCGGGTTTGCCAGCTTGTCGGCGACCGCCCCGCCGC
TCTCGCGGTGCATCACGTCGCCGACCCGGCAGCTGCGAACGACGTGGAGG
CGGCGCTGGCGGAGCGGCTGCCGGCGTGTGAGCCGGCCATGGTGACCGCC
ATGGGACCGGTACTTGCTCTGCACGTCGGTGCCGGAGCCGTCGGGGTATG
CGTCGACGTGGGAGCGTCGCCGCCAGCGTAACGTCACGGCGAAATTCGTC
GCTGATTCTCGCAGTGGCGTCACGCTGGCGGGGCTACCCGCATCGCGTGA
TCCTTTGCCAGACACTGTCGTCGTAATATTCACGTGCACGTGGCCGCGGC ATATGCCACAGTCGGATTCTGGTGACTGTGACCCTGTGTAGCCCCGACCG
AGGACGACTGGCCGGGGATGTTCCTACTGGCCGCGGCCAGTTTCACCGAT
TTCATCGGCCCTGAATCAGCGACCGCCTGGCGGACCCTGGTGCCCACCGA
CGGAGCGGTGGTGGTCCGCGATGGTGCCGGCCCGGGTTCTGAGGTGGTCG GGATGGCGCTGTACATGGA

### EmbB

The embB target region on the parent strand is a subsection of the overall 3,297 base pair embB gene. The region chosen contains all the high confidence SNPS and the majority of known embB SNPs. Forward and reverse primer locations are written in italics.

Forward Primer: 5′-CGCCGTGGTGATATTCGGC-3′ [Sequence ID No. 3]

Reverse Primer: 5′-GCACACCGTAGCTGGAGAC-3′ [Sequence ID No. 4]

CGCCGTGGTGATATTCGGCTTCCTGCTCTGGCATGTCATCGGCGCGAATT
CGTCGGACGACGGCTACATCCTGGGCATGGCCCGAGTCGCCGACCACGCC
GGCTACATGTCCAACTATTTCCGCTGGTTCGGCAGCCCGGAGGATCCCTT
CGGCTGGTATTACAACCTGCTGGCGCTGATGACCCATGTCAGCGACGCCA
GTCTGTGGATGCGCCTGCCAGACCTGGCCGCCGGGCTAGTGTGCTGGCTG
CTGCTGTCGCGTGAGGTGCTGCCCCGCCTCGGGCCGGCGGTGGAGGCCAG
CAAACCCGCCTACTGGGCGGCGGCCATGGTCTTGCTGACCGCGTGGATGC
CGTTCAACAACGGCCTGCGGCCGGAGGGCATCATCGCGCTCGGCTCGCTG
GTCACCTATGTGCTGATCGAGCGGTCCATGCGGTACAGCCGGCTCACACC
GGCGGCGCTGGCCGTCGTTACCGCCGCATTCACACTGGGTGTGCAGCCCA
CCGGCCTGATCGCGGTGGCCGCGCTGGTGGCCGGCGGCCGCCCGATGCTG
CGGATCTTGGTGCGCCGTCATCGCCTGGTCGGCACGTTGCCGTTGGTGTC
GCCGATGCTGGCCGCCGGCACCGTCATCCTGACCGTGGTGTTCGCCGACC
AGACCCTGTCAACGGTGTTGGAAGCCACCAGGGTTCGCGCCAAAATCGGG
CCGAGCCAGGCGTGGTATACCGAGAACCTGCGTTACTACTACCTCATCCT
GCCCACCGTCGACGGTTCGCTGTCGCGGCGCTTCGGCTTTTTGATCACCG
CGCTATGCCTGTTCACCGCGGTGTTCATCATGTTGCGGCGCAAGCGAATT
CCCAGCGTGGCCCGCGGACCGGCGTGGCGGCTGATGGGCGTCATCTTCGG
CACCATGTTCTTCCTGATGTTCACGCCCACCAAGTGGGTGCACCACTTCG
GGCTGTTCGCCGCCGTAGGGGCGGCGATGGCCGCGCTGACGACGGTGTTG
GTATCCCCATCGGTGCTGCGCTGGTCGCGCAACCGGATGGCGTTCCTGGC
GGCGTTATTCTTCCTGCTGGCGTTGTGTTGGGCCACCACCAACGGCTGGT
GGTATGTCTCCAGCTACGGTGTGC

### Rrs

The rrs primers target includes a subset of the 1,537 base pair rrs gene on the parent strand and some sequence outside the gene at the 3′ end as some of the target SNPs are at the 3′ end of the gene. Sequence outside the annotated gene is highlighted in grey. Forward and reverse primer locations are written in italics.

Forward Primer: 5′-CTCTGGGCAGTAACTGACGC-3′ [Sequence ID No. 5]

Reverse Primer: 5′-GAGTGTTGCCTCAGGACCC-3′ [Sequence ID No. 6]

CTCTGGGCAGTAACTGACGCTGAGGAGCGAAAGCGTGGGGAGCGAACAGG
ATTAGATACCCTGGTAGTCCACGCCGTAAACGGTGGGTACTAGGTGTGGG
TTTCCTTCCTTGGGATCCGTGCCGTAGCTAACGCATTAAGTACCCCGCCT
GGGGAGTACGGCCGCAAGGCTAAAACTCAAAGGAATTGACGGGGGCCCGC
ACAAGCGGCGGAGCATGTGGATTAATTCGATGCAACGCGAAGAACCTTAC
CTGGGTTTGACATGCACAGGACGCGTCTAGAGATAGGCGTTCCCTTGTGG
CCTGTGTGCAGGTGGTGCATGGCTGTCGTCAGCTCGTGTCGTGAGATGTT
GGGTTAAGTCCCGCAACGAGCGCAACCCTTGTCTCATGTTGCCAGCACGT
AATGGTGGGGACTCGTGAGAGACTGCCGGGGTCAACTCGGAGGAAGGTGG
GGATGACGTCAAGTCATCATGCCCCTTATGTCCAGGGCTTCACACATGCT
ACAATGGCCGGTACAAAGGGCTGCGATGCCGCGAGGTTAAGCGAATCCTT
AAAAGCCGGTCTCAGTTCGGATCGGGGTCTGCAACTCGACCCCGTGAAGT
CGGAGTCGCTAGTAATCGCAGATCAGCAACGCTGCGGTGAATACGTTCCC
GGGCCTTGTACACACCGCCCGTCACGTCATGAAAGTCGGTAACACCCGAA
GCCAGTGGCCTAACCCTCGGGAGGGAGCTGTCGAAGGTGGGATCGGCGAT
TGGGACGAAGTCGTAACAAGGTAGCCGTACCGGAAGGTGCGGCTGGATCA
CCTCCTTTCTAAGGAGCACCACGAAAACGCCCCAACTGGTGGGGCGTAGG
CCGTGAGGGGTTCTTGTCTGTAGTGGGCGAGAGCCGGGTGCATGACAACA
AAGTTGGCCACCAACACACTGTTGGGTCCTGAGGCAACACTC

### Rv0678

The rv0678 target region contains the entire 498 base pair rv0678 gene on the parent strand along with intergenic regions on either side. Sequence outside the annotated gene is highlighted in grey. Forward and reverse primer locations are written in italics.

Forward Primer: 5′-GCTCGTCCTTCACTTCGCC-3′ [Sequence ID No. 7]

Reverse Primer: 5′-ATCAGTCGTCCTCTCCGGT-3′ [Sequence ID No. 8]

GCTCGTCCTTCACTTCGCCATCGACGGTGATTCGGCAGGTGATGGAAGTG
CCGTCGCCTTGCGCGAGGATGTTGGGGGCCGCGGACGGCGCCGTGGTCTT
CAAGGTGAGCGACCGCAGGGCTGCGCCGTCGATCCGCTGTGGCTTGGCGT
CGAGGTCCAGGTAGTTGATGTTGACGTAACTACCGGAGCCGGAAACTTCG
TACTCCACCACCTTGGGGTCGAACGGCTCCGGGTCATCGGCGAAGACCTT
CGCCGTCACCAAGCATGCCTTCGGAACCAAAGAAAGTGCGGATCCGCTGC
ACCGTGAAGCCGGCGATGGCGACCACAACCAGGATGAGCAGCGGTATCCA
GGCACGCTTGAGAGTTCCAATCATCGCCCTCCGCCTCTGCCGCATGAAGT
TCACGCCGGTCTGGTGACGCATACCGAACGTCACAGATTTCAGAGTACAG
TGAAACTTGTGAGCGTCAACGACGGGGTCGATCAGATGGGCGCCGAGCCC
GACATCATGGAATTCGTCGAACAGATGGGCGGCTATTTCGAGTCCAGGAG
TTTGACTCGGTTGGCGGGTCGATTGTTGGGCTGGCTGCTGGTGTGTGATC
CCGAGCGGCAGTCCTCGGAGGAACTGGCGACGGCGCTGGCGGCCAGCAGC
GGGGGGATCAGCACCA.ATGCCCGGATGCTGATCCAATTTGGGTTCATTG
AGCGGCTCGCGGTCGCCGGGGATCGGCGCACCTATTTCCGGTTGCGGCCC
AACGCTTTCGCGGCTGGCGAGCGTGAACGCATCCGGGCAATGGCCGAACT
GCAGGACCTGGCTGACGTGGGGCTGAGGGCGCTGGGCGACGCCCCGCCGC
AGCGAAGCCGACGGCTGCGGGAGATGCGGGATCTGTTGGCATATATGGAG
AACGTCGTCTCCGACGCCCTGGGGCGATACAGCCAGCGAACCGGAGAGGA CGACTGAT

### FabG1

The fabG1 target region covers the 744 bp fabG1 gene on the parent strand along the gene promoter region (denoted in bold), targeting the high confidence SNPs located therein, and some intergenic sequence at the 3′ end. Sequence outside the annotated gene is highlighted in grey. Forward and reverse primer locations are written in italics.

Forward Primer: 5′-CTTTTGCACGCAATTGCGC-3_{′} [Sequence ID No. 9]

Reverse Primer: 5′-AGCAGTCCTGTCATGTGCG-3′ [Sequence ID No. 10]AAGTGTGC

CTTTTHCACGAATTGCGCGGYCAGTTCCACACCCTGCGGCACGTACACGT
CTTTATGTAGCGCGACATACCTGCTGCGAATTCGTAGGGCGTCAATACAC
CCGCAGCCAGGGCCTCGCTGCCCAGAAAGGGATCCGTCATGGTCGAAGTG
TGCTGAGTCACACCGACAAACGTCACGAGCGTAACCCCAGTGCGAAAGTT
CCCGCCGGAAATCGCAGCCACGTTACGCTCGTGGACATACCGATTTCGGC CCGGCCGCGGCGAGACGATAGGTTGTCGGGGTGACTGCCACAGCCACTGA
AGGGGCCAAACCCCCATTCGTATCCCGTTCAGTCCTGGTTACCGGAGGAA
ACCGGGGGATCGGGCTGGCGATCGCACAGCGGCTGGCTGCCGACGGCCAC
AAGGTGGCCGTCACCCACCGTGGATCCGGAGCGCCAAAGGGGCTGTTTGG
CGTCGAATGTGACGTCACCGACAGCGACGCCGTCGATCGCGCCTTCACGG
CGGTAGAAGAGCACCAGGGTCCGGTCGAGGTGCTGGTGTCCAACGCCGGC
CTATCCGCGGACGCATTCCTCATGCGGATGACCGAGGAAAAGTTCGAGAA
GGTCATCAACGCCAACCTCACCGGGGCGTTCCGGGTGGCTCAACGGGCAT
CGCGCAGCATGCAGCGCAACAAATTCGGTCGAATGATATTCATAGGTTCG
GTCTCCGGCAGCTGGGGCATCGGCAACCAGGCCAACTACGCAGCCTCCAA
GGCCGGAGTGATTGGCATGGCCCGCTCGATCGCCCGCGAGCTGTCGAAGG
CAAACGTGACCGCGAATGTGGTGGCCCCGGGCTACATCGACACCGATATG
ACCCGCGCGCTGGATGAGCGGATTCAGCAGGGGGCGCTGCAATTTATCCC
AGCGAAGCGGGTCGGCACCCCCGCCGAGGTCGCCGGGGTGGTCAGCTTCC
TGGCTTCCGAGGATGCGAGCTATATCTCCGGTGCGGTCATCCCGGTCGAC
GGCGGCATGGGTATGGGCCACTGACACAACACAAGGACGCACATGACAGG ACTGCT

### GyrA

The gyrA target region is a subset of the overall 2,517 bp gyrA gene on the parent strand. This target region was designed to encompass all the high confidence gyrA resistance-conferring SNPs.. Forward and reverse primer locations are written in italics.

Forward Primer: 5′-TGACAGACACGACGTTGCC-3′ [Sequence ID No. 11]

Reverse Primer: 5′-CGATCGCTAGCATGTTGGC-3′ [Sequence ID No. 12]

TGACAGACACGACGTTGCCGCCTGACGACTCGCTCGACCGGATCGAACCG
GTTGACATCGAGCAGGAGATGCAGCGCAGCTACATCGACTATGCGATGAG
CGTGATCGTCGGCCGCGCGCTGCCGGAGGTGCGCGACGGGCTCAAGCCCG
TGCATCGCCGGGTGCTCTATGCAATGTTCGATTCCGGCTTCCGCCCGGAC
CGCAGCCACGCCAAGTCGGCCCGGTCGGTTGCCGAGACCATGGGCAACTA
CCACCCGCACGGCGACGCGTCGATCTACGACAGCCTGGTGCGCATGGCCC
AGCCCTGGTCGCTGCGCTACCCGCTGGTGGACGGCCAGGGCAACTTCGGC
TCGCCAGGCAATGACCCACCGGCGGCGATGAGGTACACCGAAGCCCGGCT
GACCCCGTTGGCGATGGAGATGCTGAGGGAAATCGACGAGGAGACAGTCG
ATTTCATCCCTAACTACGACGGCCGGGTGCAAGAGCCGACGGTGCTACCC
AGCCGGTTCCCCAACCTGCTGGCCAACGGGTCAGGCGGCATCGCGGTCGG
CATGGCAACCAATATCCCGCCGCACAACCTGCGTGAGCTGGCCGACGCGG
TGTTCTGGGCGCTGGAGAATCACGACGCCGACGAAGAGGAGACCCTGGCC
GCGGTCATGGGGCGGGTTAAAGGCCCGGACTTCCCGACCGCCGGACTGAT
CGTCGGATCCCAGGGCACCGCTGATGCCTACAAAACTGGCCGCGGCTCCA
TTCGAATGCGCGGAGTTGTTGAGGTAGAAGAGGATTCCCGCGGTCGTACC
TCGCTGGTGATCACCGAGTTGCCGTATCAGGTCAACCACGACAACTTCAT
CACTTCGATCGCCGAACAGGTCCGAGACGGCAAGCTGGCCGGCATTTCCA
ACATTGAGGACCAGTCTAGCGATCGGGTCGGTTTACGCATCGTCATCGAG
ATCAAGCGCGATGCGGTGGCCAAGGTGGTGATCAATAACCTTTACAAGCA
CACCCAGCTGCAGACCAGCTTTGGCGCCAACATGCTAGCGATCG

### RpoB

The rpoB target region is a subset of the 3,519 bp rpoB gene on the parent strand. This target region was designed to encompass all the high confidence rpoB resistance-conferring SNPs. Forward and reverse primer locations are written in italics.

Forward Primer: 5′-TCATCATCAACGGGACCGAG-3′ [Sequence ID No. 13]

Reverse Primer: 5′-ACACGATCTCGTCGCTAACC-3′ [Sequence ID No. 14]

TCATCATCAACGGGACCGAGCGTGTGGTGGTCAGCCAGCTGGTGCGGTCG
CCCGGGGTGTACTTCGACGAGACCATTGACAAGTCCACCGACAAGACGCT
GCACAGCGTCAAGGTGATCCCGAGCCGCGGCGCGTGGCTCGAGTTTGACG
TCGACAAGCGCGACACCGTCGGCGTGCGCATCGACCGCAAACGCCGGCAA
CCGGTCACCGTGCTGCTCAAGGCGCTGGGCTGGACCAGCGAGCAGATTGT
CGAGCGGTTCGGGTTCTCCGAGATCATGCGATCGACGCTGGAGAAGGACA
ACACCGTCGGCACCGACGAGGCGCTGTTGGACATCTACCGCAAGCTGCGT
CCGGGCGAGCCCCCGACCAAAGAGTCAGCGCAGACGCTGTTGGAAAACTT
GTTCTTCAAGGAGAAGCGCTACGACCTGGCCCGCGTCGGTCGCTATAAGG
TCAACAAGAAGCTCGGGCTGCATGTCGGCGAGCCCATCACGTCGTCGACG
CTGACCGAAGAAGACGTCGTGGCCACCATCGAATATCTGGTCCGCTTGCA
CGAGGGTCAGACCACGATGACCGTTCCGGGCGGCGTCGAGGTGCCGGTGG
AAACCGACGACATCGACCACTTCGGCAACCGCCGCCTGCGTACGGTCGGC
GAGCTGATCCAAAACCAGATCCGGGTCGGCATGTCGCGGATGGAGCGGGT
GGTCCGGGAGCGGATGACCACCCAGGACGTGGAGGCGATCACACCGCAGA
CGTTGATCAACATCCGGCCGGTGGTCGCCGCGATCAAGGAGTTCTTCGGC
ACCAGCCAGCTGAGCCAATTCATGGACCAGAACAACCCGCTGTCGGGGTT
GACCCACAAGCGCCGACTGTCGGCGCTGGGGCCCGGCGGTCTGTCACGTG
AGCGTGCCGGGCTGGAGGTCCGCGACGTGCACCCGTCGCACTACGGCCGG
ATGTGCCCGATCGAAACCCCTGAGGGGCCCAACATCGGTCTGATCGGCTC
GCTGTCGGTGTACGCGCGGGTCAACCCGTTCGGGTTCATCGAAACGCCGT
ACCGCAAGGTGGTCGACGGCGTGGTTAGCGACGAGATCGTGT

### EthA

The ethA target region covers a subset of the 1470 base pair ethA gene on the complement strand. This section was chosen to cover the high confidence SNPs located at the 5′ end of the gene. Sequence outside the annotated gene is underlined. Forward and reverse primer locations are written italics.

Forward Primer: 5′- TGGATCCATGACCGAGCAC -3′ [Sequence ID No. 15]

Reverse Primer: 5′- GTCCAGGAGGCATTGGTGT -3′ [Sequence ID No. 16]

TGGATCCATGACCGAGCACCTCGACGTTGTCATCGTGGGCGCTGGAATCT
CCGGTGTCAGCGCGGCCTGGCACCTGCAGGACCGTTGCCCGACCAAGAGC
TACGCCATCCTGGAAAAGCGGGAATCCATGGGCGGCACCTGGGATTTGTT
CCGTTATCCCGGAATTCGCTCCGACTCCGACATGTACACGCTAGGTTTCC
GATTCCGTCCCTGGACCGGACGGCAGGCGATCGCCGACGGCAAGCCCATC
CTCGAGTACGTCAAGAGCACCGCGGCCATGTATGGAATCGACAGGCATAT
CCGGTTCCACCACAAGGTGATCAGTGCCGATTGGTCGACCGCGGAAAACC
GCTGGACCGTTCACATCCAAAGCCACGGCACGCTCAGCGCCCTCACCTGC
GAATTCCTCTTTCTGTGCAGCGGCTACTACAACTACGACGAGGGCTACTC
GCCGAGATTCGCCGGCTCGGAGGATTTCGTCGGGCCGATCATCCATCCGC
AGCACTGGCCCGAGGACCTCGACTACGACGCTAAGAACATCGTCGTGATC
GGCAGTGGCGCAACGGCGGTCACGCTCGTGCCGGCGCTGGCGGACTCGGG
CGCCAAGCACGTCACGATGCTGCAGCGCTCACCCACCTACATCGTGTCGC
AGCCAGACCGGGACGGCATCGCCGAGAAGCTCAACCGCTGGCTGCCGGAG
ACCATGGCCTACACCGCGGTACGGTGGAAGAACGTGCTGCGCCAGGCGGC
CGTGTACAGCGCCTGCCAGAAGTGGCCACGGCGCATGCGGAAGATGTTCC
TGAGCCTGATCCAGCGCCAGCTACCCGAGGGGTACGACGTGCGAAAGCAC
TTCGGCCCGCACTACAACCCCTGGGACCAGCGATTGTGCTTGGTGCCCAA
CGGCGACCTGTTCCGGGCCATTCGTCACGGGAAGGTCGAGGTGGTGACCG
ACACCATTGAACGGTTCACCGCGACCGGAATCCGGCTGAACTCAGGTCGC
GAACTGCCGGCTGACATCATCATTACCGCAACGGGGTTGAACCTGCAGCT
TTTTGGTGGGGCGACGGCGACTATCGACGGACAACAAGTGGACATCACCA
CGACGATGGCCTACAAGGGCATGATGCTTTCCGGCATCCCCAACATGGCC
TACACGGTTGGCTACACCAATGCCTCCTGGAC

### RplC

The rplC target region contains the entire 654 bp rplC gene on the parent strand along with intergenic regions on the 5′ and 3′ ends. Sequence outside the annotated gene is highlighted in grey. Forward and reverse primer locations are written in italics.

Forward Primer: 5′-AGTACAAGGACTCGCGGGA-3′ [Sequence ID No. 17]

Reverse Primer: 5′-TCGAGTGGGTACCCTGGC-3′ [Sequence ID No. 18]

AGTACAAGGAGTCGCGGGAGCACTTCGAGATGCGCACACACAAGCGGTTG
ATCGACATCATCGATCCCACGCCGAAGACCGTTGACGCGCTCATGCGCAT
CGACCTTCCGGCCAGCGTCGACGTCAACATCCAGTAGGAGATTGGACAGA
GCAATGGCACGAAAGGGCATTCTCGGTACCAAGCTGGGTATGACGCAGGT
ATTCGACGAAAGCAACAGAGTAGTACCGGTGACCGTGGTCAAGGCCGGGC
CCAACGTGGTAACCCGCATCCGCACGCCCGAACGCGACGGTTATAGCGCC
GTGCAGCTGGCCTATGGCGAGATCAGCCCACGCAAGGTCAACAAGCCGCT
GACAGGTCAGTACACCGCCGCCGGCGTCAACCCACGCCGATACCTGGCGG
AGCTGCGGCTGGACGACTCGGATGCCGCGACCGAGTACCAGGTTGGGCAA
GAGTTGACCGCGGAGATCTTCGCCGATGGCAGCTACGTCGATGTGACGGG
TACCTCCAAGGGCAAAGGTTTCGCCGGCACCATGAAGCGGCACGGCTTCC
GCGGTCAGGGCGCCAGTCACGGTGCCCAGGCGGTGCACCGCCGTCCGGGC
TCCATCGGCGGATGTGCCACGCCGGCGCGGGTGTTCAAGGGCACCCGGAT
GGCCGGGCGGATGGGCAATGACCGGGTGACCGTTCTTAACCTTTTGGTGC
ATAAGGTCGATGCCGAGAACGGCGTGCTGCTGATCAAGGGTGCGGTTCCT
GGCCGCACCGGTGGACTGGTCATGGTCCGCAGTGCGATCAAACGAGGTGA
GAAGTGATGGCTGCGCAAGAGCAGAAGACACTCAAAATCGACGTCAAGAC
GCCGGCGGGCAAGGTCGACGGCGCTATCGAGCTGCCGGCCGAGCTGTTCG
ACGTCCCGGCCAACATCGCGCTGATGCACCAGGTGGTCACCGCCCAGCGG
GCGGCGGCACGCCAGGGTACCCACTCGA

### KatG (Initial Primer Pair)

The katG target region is a subset of the 2,223 base pair katG gene, which is on the complement strand. The region was chosen to cover all high confidence SNPs. Forward and reverse primer locations are highlighted in italics.

Forward Primer: 5′- CTGTGGCCGGTCAAGAAGA -3′ [Sequence ID No.19]

Reverse Primer: 5′- TGCCCGGATCTGGCTCTTA -3′ [Sequence ID No.33]

CTGTGGCCGGTCAAGAAGAAGTACGGCAAGAAGCTCTCATGGGCGGACCT
GATTGTTTTCGCCGGCAACTGCGCGCTGGAATCGATGGGCTTCAAGACGT
TCGGGTTCGGCTTCGGCCGGGTCGACCAGTGGGAGCCCGATGAGGTCTAT
TGGGGCAAGGAAGCCACCTGGCTCGGCGATGAGCGTTACAGCGGTAAGCG
GGATCTGGAGAACCCGCTGGCCGCGGTGCAGATGGGGCTGATCTACGTGA
ACCCGGAGGGGCCGAACGGCAACCCGGACCCCATGGCCGCGGCGGTCGAC
ATTCGCGAGACGTTTCGGCGCATGGCCATGAACGACGTCGAAACAGCGGC
GCTGATCGTCGGCGGTCACACTTTCGGTAAGACCCATGGCGCCGGCCCGG
CCGATCTGGTCGGCCCCGAACCCGAGGCTGCTCCGCTGGAGCAGATGGGC
TTGGGCTGGAAGAGCTCGTATGGCACCGGAACCGGTAAGGACGCGATCAC
CAGCGGCATCGAGGTCGTATGGACGAACACCCCGACGAAATGGGACAACA
GTTTCCTCGAGATCCTGTACGGCTACGAGTGGGAGCTGACGAAGAGCCCT
GCTGGCGCTTGGCAATACACCGCCAAGGACGGCGCCGGTGCCGGCACCAT
CCCGGACCCGTTCGGCGGGCCAGGGCGCTCCCCGACGATGCTGGCCACTG
ACCTCTCGCTGCGGGTGGATCCGATCTATGAGCGGATCACGCGTCGCTGG
CTGGAACACCCCGAGGAATTGGCCGACGAGTTCGCCAAGGCCTGGTACAA
GCTGATCCACCGAGACATGGGTCCCGTTGCGAGATACCTTGGGCCGCTGG
TCCCCAAGCAGACCCTGCTGTGGCAGGATCCGGTCCCTGCGGTCAGCCAC
GACCTCGTCGGCGAAGCCGAGATTGCCAGCCTTAAGAGCCAGATCCGGGC A

### KatG - Redesigned

The katG target region is a subset of the 2,223 bp katG gene, which is on the complement strand. The region was chosen to cover all the high confidence SNPs. Forward and reverse primer locations are written in italics.

Forward Primer: 5′- CTGTGGCCGGTCAAGAAGA -3′ [Sequence ID No. 19]

Reverse Primer: 5′- GGATCTGGCTCTTAAGGCTGG -3′ [Sequence ID No. 20]

CTGTGGCCGGTCAAGAAGAAGTACGGCAAGAAGCTCTCATGGGCGGACCT
GATTGTTTTCGCCGGCAACTGCGCGCTGGAATCGATGGGCTTCAAGACGT
TCGGGTTCGGCTTCGGCCGGGTCGACCAGTGGGAGCCCGATGAGGTCTAT
TGGGGCAAGGAAGCCACCTGGCTCGGCGATGAGCGTTACAGCGGTAAGCG
GGATCTGGAGAACCCGCTGGCCGCGGTGCAGATGGGGCTGATCTACGTGA
ACCCGGAGGGGCCGAACGGCAACCCGGACCCCATGGCCGCGGCGGTCGAC
ATTCGCGAGACGTTTCGGCGCATGGCCATGAACGACGTCGAAACAGCGGC
GCTGATCGTCGGCGGTCACACTTTCGGTAAGACCCATGGCGCCGGCCCGG
CCGATCTGGTCGGCCCCGAACCCGAGGCTGCTCCGCTGGAGCAGATGGGC
TTGGGCTGGAAGAGCTCGTATGGCACCGGAACCGGTAAGGACGCGATCAC
CAGCGGCATCGAGGTCGTATGGACGAACACCCCGACGAAATGGGACAACA
GTTTCCTCGAGATCCTGTACGGCTACGAGTGGGAGCTGACGAAGAGCCCT
GCTGGCGCTTGGCAATACACCGCCAAGGACGGCGCCGGTGCCGGCACCAT
CCCGGACCCGTTCGGCGGGCCAGGGCGCTCCCCGACGATGCTGGCCACTG
ACCTCTCGCTGCGGGTGGATCCGATCTATGAGCGGATCACGCGTCGCTGG
CTGGAACACCCCGAGGAATTGGCCGACGAGTTCGCCAAGGCCTGGTACAA
GCTGATCCACCGAGACATGGGTCCCGTTGCGAGATACCTTGGGCCGCTGG
TCCCCAAGCAGACCCTGCTGTGGCAGGATCCGGTCCCTGCGGTCAGCCAC
GACCTCGTCGGCGAAGCCGAGATTGCCAGCCTTAAGAGCCAGATCCGGGC A

### GidB

The gidB target region contains the entire 675 bp gidB gene on the parent strand along with intergenic sequence on the 5′ and 3′ ends. Sequence outside the annotated gene is highlighted in grey. Forward and reverse primer locations are written in italics.

Forward Primer: 5′-TGACACAGACCTCACGAGC-3′ [Sequence ID No. 21]

Reverse Primer: 5′-GCCCTTCTGATTCGCGATG-3′ [Sequence ID No. 22]

TGACACAGACCTCAGGAGCCGGCGGAGTGCGTAATGTCTCCGATCGAGCC
CGCGGCGTCTGCGATCTTCGGACCGCGGCTTGGCCTTGCTCGGCGGTACG
CCGAAGCGTTGGCGGGACCCGGTGTGGAGCGGGGGCTGGTGGGACCCCGC
GAAGTCGGTAGGCTATGGGACCGGCATCTACTGAACTGCGCCGTGATCGG
TGAGCTCCTCGAACGCGGTGACCGGGTCGTGGATATCGGTAGCGGAGCCG
GGTTGCCGGGCGTGCCATTGGCGATAGCGCGGCCGGACCTCCAGGTAGTT
CTCCTAGAACCGCTACTGCGCCGCACCGAGTTTCTTCGAGAGATGGTGAC
AGATCTGGGCGTGGCCGTTGAGATCGTGCGGGGGCGCGCCGAGGAGTCCT
GGGTGCAGGACCAATTGGGCGGCAGCGACGCTGCGGTGTCACGGGCGGTG
GCCGCGTTGGACAAGTTGACGAAATGGAGCATGCCGTTGATACGGCCGAA
CGGGCGAATGCTCGCCATCAAAGGCGAGCGGGCTCACGACGAAGTACGGG
AGCACCGGCGTGTGATGATCGCATCGGGCGCGGTTGATGTCAGGGTGGTG
ACATGTGGCGCGAACTATTTGCGTCCGCCCGCGACCGTGGTGTTCGCACG
ACGTGGAAAGCAGATCGCCCGAGGGTCGGCACGGATGGCGAGTGGAGGGA
CGGCGTGAGTGCTCCGTGGGGCCCGGTGGCCGCTGGACCGTCCGCGCTCG
TAAGGTCGGGCCAGGCTTCAACTATCGAACCATTCCAGCGGGAAATGACA
CCACCGACACCGACGCCTGAGGCCGCGCACAATCCGACGATGAATGTTTC
ACGTAGAAACATCGACAGAATTCGACACCCCCATCGGCGCTGCAGCAGAA
CGTGCGATGCGGGTCCTGCACACCACCCACGAGCCGCTGCAGCGGCCGGG
TCGACGCCGGGTGCTCACCATCGCGAATCAGAAGGGC

### InhA

The inha target region contains a subset of the inhA 810 bp gene on the parent strand along with the promoter region, denoted in bold, to cover all the high confidence SNPs in the gene and promotor. Sequence outside the annotated gene is highlighted in grey. Forward and reverse primer locations are highlighted in italics.

Forward Primer: 5′-GGGCGCTGCAATTTATCCC-3′ [Sequence ID No. 23]

Reverse Primer: 5′-GGCGTAGATGATGTCACCC-3′ [Sequence ID No. 24]

GGGCGCTGCAATTTATCCCAGCGAAGCGGGTCGGCACCCCCGCCGAGGTC
GCCGGGGTGGTCAGCTTCCTGGCTTCCGAGGATGCGAGCTATATCTCCGG
TGCGGTCATCCCGGTCGACGGCGGCATGGGTATGGGCCACTGACACAACA CAAGGACGCACATGACAGGACTGCTGGACGGCAAACGGATTCTGGTTAGC
GGAATCATCACCGACTCGTCGATCGCGTTTCACATCGCACGGGTAGCCCA
GGAGCAGGGCGCCCAGCTGGTGCTCACCGGGTTCGACCGGCTGCGGCTGA
TTCAGCGCATCACCGACCGGCTGCCGGCAAAGGCCCCGCTGCTCGAACTC
GACGTGCAAAACGAGGAGCACCTGGCCAGCTTGGCCGGCCGGGTGACCGA
GGCGATCGGGGCGGGCAACAAGCTCGACGGGGTGGTGCATTCGATTGGGT
TCATGCCGCAGACCGGGATGGGCATCAACCCGTTCTTCGACGCGCCCTAC
GCGGATGTGTCCAAGGGCATCCACATCTCGGCGTATTCGTATGCTTCGAT
GGCCAAGGCGCTGCTGCCGATCATGAACCCCGGAGGTTCCATCGTCGGCA
TGGACTTCGACCCGAGCCGGGCGATGCCGGCCTACAACTGGATGACGGTC
GCCAAGAGCGCGTTGGAGTCGGTCAACAGGTTCGTGGCGCGCGAGGCCGG
CAAGTACGGTGTGCGTTCGAATCTCGTTGCCGCAGGCCCTATCCGGACGC
TGGCGATGAGTGCGATCGTCGGCGGTGCGCTCGGCGAGGAGGCCGGCGCC
CAGATCCAGCTGCTCGAGGAGGGCTGGGATCAGCGCGCTCCGATCGGCTG
GAACATGAAGGATGCGACGCCGGTCGCCAAGACGGTGTGCGCGCTGCTGT
CTGACTGGCTGCCGGCGACCACGGGTGACATCATCTACGCC

### Rrl

The rrl target region is a subsection of the overall 3,138 bp rrl gene on the parent strand, targeting all the high confidence SNPs. Forward and reverse primer locations are written in italics.

Forward Primer: 5′-GGTCCGTGCGAAGTCGC-3′ [Sequence ID No. 25]

Reverse Primer: 5′-TGAACCCGTGTTCTGCGG-3′ [Sequence ID No. 26]

GGTCCGTGCGAAGTCGCAAGACGATGTATACGGACTGACGCCTGCCCGGT
GCTGGAAGGTTAAGAGGACCCGTTAACCCGCAAGGGTGAAGCGGAGAATT
TAAGCCCCAGTAAACGGCGGTGGTAACTATAACCATCCTAAGGTAGCGAA
ATTCCTTGTCGGGTAAGTTCCGACCTGCACGAATGGCGTAACGACTTCTC
AACTGTCTCAACCATAGACTCGGCGAAATTGCACTACGAGTAAAGATGCT
CGTTACGCGCGGCAGGACGAAAAGACCCCGGGACCTTCACTACAACTTGG
TATTGATGTTCGGTACGGTTTGTGTAGGATAGGTGGGAGACTGTGAAACC
TCGACGCCAGTTGGGGCGGAGTCGTTGTTGAAATACCACTCTGATCGTAT
TGGGCATCTAACCTCGAACCCTGAATCGGGTTTAGGGACAGTGCCTGGCG
GGTAGTTTAACTGGGGCGGTTGCCTCCTAAAATGTAACGGAGGCGCCCAA
AGGTTCCCTCAACCTGGACGGCAATCAGGTGGCGAGTGTAAATGCACAAG
GGAGCTTGACTGCGAGACTTACAAGTCAAGCAGGGACGAAAGTCGGGATT
AGTGATCCGGCACCCCCGAGTGGAAGGGGTGTCGCTCAACGGATAAAAGG
TACCCCGGGGATAACAGGCTGATCTTCCCCAAGAGTCCATATCGACGGGA
TGGTTTGGCACCTCGATGTCGGCTCGTCGCATCCTGGGGCTGGAGCAGGT
CCCAAGGGTTGGGCTGTTCGCCCATTAAAGCGGCACGCGAGCTGGGTTTA
GAACGTCGTGAGACAGTTCGGTCTCTATCCGCCGCGCGCGTCAGAAACTT
GAGGAAACCTGTCCCTAGTACGAGAGGACCGGGACGGACGAACCTCTGGT
GCACCAGTTGTCCCGCCAGGGGCACCGCTGGATAGCCACGTTCGGTCAGG
ATAACCGCTGAAAGCATCTAAGCGGGAAACCTTCTCCAAGATCAGGTTTC
TCACCCACTTGGTGGGATAAGGCCCCCCGCAGAACACGGGTTCA

### PncA

The pncA target region contains the entire 561 base pair pncA gene on the complement strand along with intergenic regions at the 5′ and 3′ ends. Sequence outside the annotated gene is highlighted in grey. Forward and reverse primer locations are written in italics.

Forward Primer: 5′-TCACCGGACGGATTTGTCG-3′ [Sequence ID No. 27]

Reverse Primer: 5′-TCCAGATCGCGATGGAACG-3′ [Sequence ID No. 28]

TCACCGGACGGATTTGTCGCTCACTACATCACCGGCGTGATCTATCCCGC
CGGTTGGGTGGCCGCCGCTCAGCTGGTCATGTTCGCGATCGTCGCGGCGT
CATGGACCCTATATCTGTGGCTGCCGCGTCGGTAGGCAAACTGCCCGGGC
AGTCGCCCGAACGTGGACGTATGCGGGCGTTGATCATCGTCGACGTGCAG
AACGACTTCTGCGAGGGTGGCTCGCTGGCGGTAACCGGTGGCGCCGCGCT
GGCCCGCGCCATCAGCGACTACCTGGCCGAAGCGGCGGACTACCATCACG
TCGTGGCAACCAAGGACTTCCACATCGACCCGGGTGACCACTTCTCCGGC
ACACCGGACTATTCCTCGTCGTGGCCACCGCATTGCGTCAGCGGTACTCC
CGGCGCGGACTTCCATCCCAGTCTGGACACGTCGGCAATCGAGGCGGTGT
TCTACAAGGGTGCCTACACCGGAGCGTACAGCGGCTTCGAAGGAGTCGAC
GAGAACGGCACGCCACTGCTGAATTGGCTGCGGCAACGCGGCGTCGATGA
GGTCGATGTGGTCGGTATTGCCACCGATCATTGTGTGCGCCAGACGGCCG
AGGACGCGGTACGCAATGGCTTGGCCACCAGGGTGCTGGTGGACCTGACA
GCGGGTGTGTCGGCCGATACCACCGTCGCCGCGCTGGAGGAGATGCGCAC
CGCCAGCGTCGAGTTGGTTTGCAGCTCCTGATGGCACCGCCGAACCGGGA
TGAACTGTTGGCGGCGGTGGAGCGCTCGCCGCAAGCGGCCGCCGCGCACG
ACCGCGCCGGCTGGGTCGGGTTGTTCACCGGTGACGCGCGGGTCGAAGAC
CCGGTGGGTTCGCAGCCGCAGGTGGGGCATGAGGCCATCGGCCGCTTCTA
CGACACCTTCATCGGGCCGCGGGATATCACGTTCCATCGCGATCTGGA

### RpsL

The rpsL target region contains the entire 375 bp rpsL gene on the parent strand along with intergenic regions at the 5′ and 3′ ends. Sequence outside the annotated gene is highlighted in grey. Forward and reverse primer locations are written in italics.

Forward Primer: 5′-GCGGCGGGTATTGTGGTT-3′ [Sequence ID No. 29]

Reverse Primer: 5′-TAACCGGCGCTTCTCACC-3′ [Sequence ID No. 30]

GCGGCGGGTATTGTGGTTGCTCGTGCCTGGCGGCTTACGCTTGATGTAGG
GGCGTGGATGCCGGGCCAATTCGCATGTCCGCGATGCCTCGGATGAGACG
AATCGAGTTTGAGGCAAGCTATGCGACACACCCGGCCGCGGGTAACCGTG
GCGGGGCATGGCCGACAAACAGAACGTGAAAGCGCCCAAGATAGAAAGCC
GGTAGATGCCAACCATCCAGCAGCTGGTCCGCAAGGGTCGTCGGGACAAG
ATCAGTAAGGTCAAGACCGCGGCTCTGAAGGGCAGCCCGCAGCGTCGTGG
TGTATGCACCCGCGTGTACACCACCACTCCGAAGAAGCCGAACTCGGCGC
TTCGGAAGGTTGCCCGCGTGAAGTTGACGAGTCAGGTCGAGGTCACGGCG
TACATTCCCGGCGAGGGCCACAACCTGCAGGAGCACTCGATGGTGCTGGT
GCGCGGCGGCCGGGTGAAGGACCTGCCTGGTGTGCGCTACAAGATCATCC
GCGGTTCGCTGGATACGCAGGGTGTCAAGAACCGCAAACAGGCACGCAGC
CGTTACGGCGCTAAGAAGGAGAAGGGCTGATGCCAGCAAGGGGCCCGCGC
CCAAGCGTCCGTTGGTCAACGACCCGGTCTACGGATCGCAGTTGGTCAGG
GAGTTGGTGAAGAAGGTTCTGTTGAAGGGGAAAAAATCGCTGGCCGAGCG
CATTGTTTATGGTGCGCTTGAGCAAGCTCGCGACAAGACCGGCACCGATC
CGGTGATCACCCTCAAGCGGGCTCTCGACAATGTCAAACCCGCCCTGGAG
GTGCGCAGCCGTCGCGTCGGCGGCGCGACCTATCAGGTGCCTGTCGAGGT
GCGCCCCGACCGGTCGACCACGCTGGCGCTGCGCTGGCTCGTCGGCTACT
GGCGGCAACGCCGTGAGAAGACGATGATCGAGCGCCTGGCAAATGGAGAT
CCTGGATGCCAGCAATGGCCTTGGGGCCTCCGTCAAGCGGCGTGAGGACA
CCCACAAGATGGCCGAGGCGAACCGAGCCTTTGCGCATTATCGCTGGTGA GAAGCGCCGGTTA

### TlyA

The tlyA target region contains the entire 807 base pair tfyA gene on the parent strand along with intergenic regions at the 5′ and 3′ ends. Sequence outside the annotated gene is highlighted in grey. Forward and reverse primer locations are written in italics.

Forward Primer: 5′-CGTTGATGCGCAGCGATC-3′ [Sequence ID No. 31]

Reverse Primer: 5′-GGTCTCGGTGGCTTCGTC-3′ [Sequence ID No. 32]

CGTTGATGCGCAGCGATCATCCGGTGACTAGCGTAGGAACGCAATGACCA
TCGATCCTGACCAGATCCGTGCCGAAATCGACGCCCTACTTGCTTCGCTG
CCCGACCCCGCCGAGCCGAGAACGGACCGTCTCTGGCCGAACTCGAAGGC
ATCGCACGTCGTCTTTCCGAGGCGCACGSGGTGTTGGCCGCCCTGGAGTC
GGCGGAGAAGGGTTGAGTGCGGCTGGCACGACGTGCCCGCGTTGACGCCG
AGCTAGTCCGGCGGGGCCTGGCGCGATCACGTCAACAGGCCGCGGAGTTG
ATCGGCGCCGGCAAGGTGCGCATCGACGGGCTGCCGGCGGTCAAGCCGGC
CACCGCCGTGTCCGACACCACCGCGCTGACCGTGGTGACCGACAGTGAAC
GCGCCTGGGTATCGCGCGGAGCGCACAAACTAGTCGGTGCGCTGGAGGCG
TTCGCGATCGCGGTGGCGGGCCGGCGCTGTCTGGACGCGGGCGCATCGAC
CGGTGGGTTCACCGAAGTACTGCTGGACCGTGGTGCCGCCCACGTGGTGG
CCGCCGATGTCGGATACGGCCAGCTGGCGTGGTCGCTGCGCAACGATCCT
CGGGTGGTGGTCCTCGAGCGGACCAACGCACGTGGCCTCACACCGGAGGC
GATCGGCGGTCGCGTCGACCTGGTAGTGGCCGACCTGTCGTTCATCTCGT
TGGCTACCGTGTTGCCCGCGCTGGTTGGATGCGCTTCGCGCGACGCCGAT
ATCGTTCCACTGGTGAAGCCGCAGTTTGAGGTGGGGAAAGGTCAGGTCGG
CCCCGGTGGGGTGGTCCATGACCCGCAGTTGCGTGCGCGGTCGGTGCTCG
CGGTCGCGCGGCGGGCACAGGAGCTGGGCTGGCACAGCGTCGGCGTCAAG
GCCAGCCCGCTGCCGGGCCCATCGGGCAATGTCGAGTACTTCCTGTGGTT
GCGCACGCAGACCGACCGGGCATTGTCGGCCAAGGGATTGGAGGATGCGG
TGCACCGTGCGATTAGCGAGGGCCCGTAGTGACCGCTCATCGCAGTGTTC
TGCTGGTCGTCCACACCGGGCGCGACGAAGCCACCGAGACC

### Advantages

The present disclosure provides a means of accurately and rapidly identifying the presence of multiple drug resistance mutations in a sample from a patient with suspected or confirmed Tuberculosis. Such information informs decisions regarding drug administration, and allows a tailored regimen to be determined for the patient depending upon the identified mutations. Furthermore, the disclosed methods can be successfully carried out on samples taken directly from patients, such as sputum, thereby adding to their potential for use in lower and middle income and developing countries. The development of optimised primers for this purpose means the advantages of using a multiplex assay can be realised. The disclosed methods are highly sensitive (<100 MTB cells), rapid (taking approximately 8 hours) and can detect a broad range of mutations, and thus represent a major improvement over current culture, molecular (e.g. GenoType MTBDRsl line probe assay) and tNGS based tests. This allows the correct treatment pathway to be determined and for patients to commence treatment promptly and not be lost to follow-up (a major problem in developing countries). This reduces the spread of disease and helps prevent the development of drug-resistant bacterial strains.

### General

Wherever the term ‘comprising’ is used herein we also contemplate options wherein the terms ‘consisting of or ‘consisting essentially of are used instead. In addition, any and all liquid compositions described herein can be aqueous solutions. Note too that whenever the phrase “one or more” is used for a range, for example in relation to a number of sequences W, X, Y and Z (“one or more of SEQ ID Nos. W, X, Y and Z”) this is a disclosure of each value alone (SEQ ID No. W; SEQ ID No. X; SEQ ID No. Y; SEQ ID No. Z), or in combination, e.g. SEQ ID Nos. W and X and SEQ ID No. Y and Z). Similarly, whenever the phrase “one or more” is used in relation to a range of pairs, for example in relation to a number of pairs of sequences (“one or more of SEQ ID Nos. W and X; and Y and Z”) this is a disclosure of each pair alone (SEQ ID No. W and X) or in combination (e.g. SEQ ID Nos. W and X and SEQ ID Nos. Y and Z).

The following Examples are provided to illustrate embodiments of the present invention and should not be construed as limiting thereof.

### Example 1

A study was conducted using sputum spiked with well characterized M. tuberculosis isolates (whole-genome sequence and culture confirmed resistance profiles) to evaluate the developed primers and method. DNA was extracted on the MagNA Pure Compact, PCR amplified in 3 multiplex reactions per sample, pooled, washed, barcoded, and sequenced on the MinION in batches of 80 as described below.

DNA Extraction:
1. In a Microbiological Class II Safety Cabinet (MSC-II) unseal liquid clinical sample and aliquot 750 µL to a fresh 1.5 mL Eppendorf tube with screw cap.
2. In MSC-II load sample Eppendorf tubes into an aerosol-sealable centrifuge rotor.
3. Centrifuge 750 µL clinical sputum sample at 15,000 g for 5 min, after which the centrifuge rotor is returned to the MSC-II and samples removed.
4. In MSC-II carefully remove supernatant and resuspend pellet in 700 µL MagNA Pure Bacterial Lysis Buffer (BLB) [Roche Life Science].
5. In MSC-II transfer 700 µL of resuspended samples to bead-beating tubes with screw cap (Lysing Matrix E tubes from MP Biomedical).
6. In MSC-II bead-beat samples in a FastPrep homogenizer at maximum speed for 45 seconds.
7. Repeat Step 6.
8. In MSC-II load bead-beating tubes into an aerosol-sealable centrifuge rotor.
9. Spin down bead-beating tubes at maximum speed for 2 minutes.
10. Return centrifuge rotor to the MSC-II and gently remove bead-beating tubes.
11. In MSC-II transfer 230 µL clear supernatant in two 200 µL batches to a clean MagNA Pure sample tube. Add 20 µL Proteinase K to sample.
12. In MSC-II incubate samples on heat block for 5 minutes at 65° C. vortexing in the MSC-II every 30 seconds.
13. Transfer incubated samples to MagNA Pure compact and perform automated extraction.
14. On completion of automated extraction return elute tubes to MSC-II for Multiplex PCR preparation.

Multiplex PCR:

1. Prepare 3 multiplex 10x primer mixes as follows:

Group 1 10x Primer Mix

| Primer | Volume Added (µL) | Final Concentration |
|---|---|---|
| 100 µM eis FW | 10 | 2 µM |
| 100 µM eis RV | 10 | 2 µM |
| 100 µM embB FW | 10 | 2 µM |
| 100 µM embB RV | 10 | 2 µM |
| 100 µM rrs FW | 10 | 2 µM |
| 100 µM rrs RV | 10 | 2 µM |
| 100 µM rv0678 FW | 10 | 2 µM |
| 100 µM rv0678 RV | 10 | 2 µM |
| 100 µM fabG1 FW | 10 | 2 µM |
| 100 µM fabG1 RV | 10 | 2 µM |
| Nuclease-Free H₂O | 400 | |
| Total Volume | 500 | |

Group 2 10x Primer Mix

| Primer Pair | Volume Added (µL) | Final Concentration |
|---|---|---|
| 100 µM gyrA FW | 10 | 2 µM |
| 100 µM gyrA RV | 10 | 2 µM |
| 100 µM rpoB FW | 10 | 2 µM |
| 100 µM rpoB RV | 10 | 2 µM |
| 100 µM ethA FW | 10 | 2 µM |
| 100 µM ethA RV | 10 | 2 µM |
| 100 µM rplC FW | 10 | 2 µM |
| 100 µM rplC RV | 10 | 2 µM |
| 100 µM katG FW | 10 | 2 µM |
| 100 µM katG RV | 10 | 2 µM |
| Nuclease-Free H₂O | 400 | |
| Total Volume | 500 | |

Group 3 10x Primer Mix

| Primer Pair | Volume Added (µL) | Final Concentration |
|---|---|---|
| 100 µM gidB FW | 10 | 2 µM |
| 100 µM gidB RV | 10 | 2 µM |
| 100 µM inhA FW | 10 | 2 µM |
| 100 µM inhA RV | 10 | 2 µM |
| 100 µM rrl FW | 10 | 2 µM |
| 100 µM rrl RV | 10 | 2 µM |
| 100 µM rpsL FW | 10 | 2 µM |
| 100 µM rpsL RV | 10 | 2 µM |
| 100 µM pncA FW | 10 | 2 µM |
| 100 µM pncA RV | 10 | 2 µM |
| 100 µM tlyA FW | 15 | 3 µM |
| 100 µM tlyA RV | 15 | 3 µM |
| Nuclease-Free H₂O | 370 | |
| Total Volume | 500 | |

2. In MSC-II mix PCR Master Mix (Qiagen Multiplex PCR kit) for each multiplex primer group in the following ratio per sample:

| Reagent | Volume per Sample (µL) |
|---|---|
| 2x Qiagen Multiplex Master Mix | 25 |
| 10x Primer Mix | 5 |
| 5x Q-Solution | 10 |
| Nuclease-Free Water | 5 |

3. In MSC-II add 45 µL mastermix to 0.2 mL thin-walled PCR tubes.

a. Each sample requires three tubes, one for each Multiplex Primer Group.

4. In MSC-II carefully add 5 µL extracted DNA to PCR tubes.

5. In MSC-II seal PCR tubes tightly and vortex.

6. In MSC-II briefly spin down PCR tubes and remove bubbles.

7. Load PCR tubes into a thermocycler and run an amplification protocol with the following parameters:

| Step | Time (mm:ss) | Temperature (°C) | Cycles |
|---|---|---|---|
| Heat Activation | 20:00 | 95 | 1 |
| Denaturation | 00:30 | 95 | 35 |
| Annealing | 01:30 | 60 | |
| Extension | 01:30 | 72 | |
| Final Extension | 10:00 | 72 | 1 |

8. Carefully remove PCR tubes and return to MSC-II.

9. In MSC-II transfer PCR product to clean PCR tubes.

10. Submerge clean PCR tubes in a 1:16 dilution of Bioguard for minimum 30 seconds for removal from CL3.

The three multiplex reactions for each sample are then pooled as follows:
1. Mix Qubit High Sensitivity assay buffer according to manufacturer specifications for each sample Multiplex Group.
   a. 200 µL Qubit Buffer + 1 µL Qubit Dye per sample
2. In a clear flat-bottomed 96-well plate aliquot 198 µL of mixed Qubit solution to each well.
3. Add 2 µL of each multiplex group template so each well has a single template.
4. Analyze plate on a Promega QuantiFlor or similar plate reader.
5. Using quantification results, pool the 3 sample multiplex groups in equimolar concentrations to a total of 1 µg.
   a. In case pooled sample total volume is below 45 µL normalize volume of all samples to 100 µL using Nuclease-Free H₂O
   b. If there is insufficient DNA for a pooled total of 1 µg, equimolar pool at a lower concentration but in a max volume of 100 µl

The pooled samples were then prepared for nanopore sequencing as follows:

### End Prep

1. Transfer 45 µL of pooled DNA to a thin-walled PCR plate

2. Add following reagents to the DNA

| Reagent | Volume per Sample (µL) |
|---|---|
| Template DNA (<1,000 ng) | 45 |
| Ultra II End-Prep Buffer | 7 |
| Ultra II End-Prep Enzyme Mix | 3 |
| Nuclease Free H₂O | 5 |
| Total | 60 |

3. Mix by pipette

4. Spin down tube and incubate for 5 minutes at 20° C. followed by 5 minutes at 65° C.

5. Transfer samples to a clean 96-well plate

6. Perform a 1x bead wash by adding 60 µL AMPure XP Beads

7. Incubate sample for 5 minutes on a hula mixer

8. Briefly spin down plate

9. Place plate on magnet-rack and let incubate for 5 minutes

10. Remove supernatant

11. Wash bead pellet with 180 µL 70% ethanol

12. Remove supernatant

13. Wash bead pellet with 180 µL 70% ethanol

14. Remove supernatant

15. Briefly spin down plate and return to magnet-rack

16. Remove residual supernatant

17. Air dry pellet for approximately 30 seconds

18. Resuspend pellet in 31 µL nuclease free H₂O

19. Incubate samples for 2 minutes at room temperature

20. Return plate to magnet-rack and pellet beads for 2 minutes

21. Carefully remove eluted supernatant and transfer 30 µL to a clean 96-well plate

### Barcode Adapter Ligation

1. In a fresh plate add the following reagents in order per sample.
   a. 15 µL End-Prepped DNA
   b. 10 µL Barcode Adapter (BCA)
   c. 25 µL Blunt/TA Ligase Master Mix
2. Mix by pipetting.
3. Briefly spin down plate.
4. Incubate at room temperature for 10 minutes
5. Perform 0.8x bead wash (30 µL) using AMPure XP beads as described above
6. Resuspend pellet in 25 µL nuclease free H₂O
7. Incubate samples for 2 minutes at room temperature
8. Return plate to magnet-rack and pellet beads for 2 minutes
9. Carefully remove eluted supernatant and transfer to a clean 96-well plate.

### Barcoding PCR

1. In a thin-walled PCR plate combine the following:

| Reagent | Volume per Sample (µL) |
|---|---|
| Adapter Ligated Template DNA | 4 |
| 10 µM PCR Barcode | 1 |
| 2x LongAmp Taq MasterMix | 25 |
| Nuclease Free H₂O | 20 |
| Total | 50 |

2. Briefly vortex

3. Spin down samples

4. PCR amplify using the following cycling conditions

| Cycle Step | Temperature (°C) | Time (mm:ss) | Cycles |
|---|---|---|---|
| Initial Denaturation | 95 | 03:00 | 1 |
| Denaturation | 95 | 00:15 | 15 |
| Annealing | 62 | 00:15 | |
| Extension | 65 | 01:30 | |
| Final Extension | 65 | 05:00 | 1 |
| Hold | 4 | ∞ | N/A |

5. Perform 0.8x bead wash (40 µL) using AMPure XP beads as described above

6. Resuspend pellet in 45 µL nuclease free H₂O

7. Incubate samples for 2 minutes at room temperature

8. Return plate to magnet-rack and pellet beads for 2 minutes

9. Carefully remove eluted supernatant and transfer to a clean 96-well plate.

10. Quantify as described above

11. Pool each barcoded sample equimolar into a fresh 1.5 mL Eppendorf

12. Perform 0.8x bead wash using AMPure XP beads on pooled samples as described above and resuspend in 45 µL nuclease free H₂O

### DNA End-Prep

1. In a 0.2 mL thin walled PCR tube combine the following:

| Reagent | Volume (µL) |
|---|---|
| Pooled Barcoded DNA (1,000 ng) + Nuclease Free H₂O | 50 |
| Ultra II End-Prep Buffer | 7 |
| Ultra II End-Prep Enzyme Mix | 3 |
| Total | 60 |

2. Vortex and briefly spin down

3. Incubate for 5 minutes at 20° C. followed by 5 minutes at 65° C.

4. Transfer sample to a clean 1.5 mL Eppendorf

5. Perform a 0.8x bead wash (48 µL) using AMPure XP beads as described above

6. Resuspend pellet in 61 µL nuclease free H₂O

7. Incubate samples for 2 minutes at room temperature

8. Return plate to magnet-rack and pellet beads for 2 minutes

9. Carefully remove eluted supernatant and transfer to a clean 1.5 mL Eppendorf.

### Adapter Ligation:

1. Thaw and spin down Adapter Mix (AMX), T4 Ligase, Ligation Buffer (LNB), and Elution Buffer (EB) (Oxford Nanopore Technologies Ligation Sequencing Kit SQK-LSK109).

2. Place thawed and vortexed reagents on ice

3. Thaw one tube of Short Fragment Buffer (SFB) at room temperature

a. Vortex and spin down before placing on ice

4. Mix the following in a 1.5 mL Eppendorf in order:

| Reagent | Volume (µL) |
|---|---|
| End-Prepped DNA | 60 |
| Ligation Buffer (LNB) | 25 |
| NEBNext Quick T4 DNA Ligase | 10 |
| Adapter Mix (AMX) | 5 |
| Total | 100 |

5. Gently mix tube by flicking and spin down

6. Incubate for 10 minutes at room temperature

7. Perform a 0.6x bead wash (60 µL) using AMPure XP beads

8. Incubate samples for 5 minutes on a hula mixer

9. Briefly spin down samples

10. Place tube on magnet-rack and let incubate for 5 minutes

11. Remove supernatant

12. Resuspend pellet in 125 µL SFB

13. Place tube on magnet-rack and let incubate for 10 minutes

14. Carefully remove supernatant

15. Resuspend pellet in 125 µL SFB

16. Place tube on magnet-rack and let incubate for 10 minutes

17. Carefully remove supernatant

18. Briefly spin down tube and return to magnet-rack

19. Remove residual supernatant

20. Air dry pellet for approximately 30 seconds

21. Resuspend pellet in 15 µL EB

22. Incubate at room temperature for 10 minutes

23. Place tube on magnet-rack until elute is clear and colourless

24. Carefully remove and retain 15 µL eluted supernatant in clean 1.5 mL Eppendorf

25. Perform Qubit HS Assay on 1 µL elute

Sequencing library loading on MinION
1. Perform MinION loading according to Oxford Nanopore Manufacturer protocols
   a. Load between 100 and 150 fmol of DNA as calculated using the Qubit quantification
   i. fmols can be calculated easily from ng using the following website: http://molbiol.edu.ru/eng/scripts/01_07.html

Resistance to first- and second-line anti-TB drugs was identified using the ONT Epi2Me FastQ TB Resistance Profile pipeline. Wild-type and mutant nucleotides were reported for all drug resistance associated SNP sites detected within the PCR product fastQ sequences. The presence of SNPs in specific target genes indicated resistance to specific anti-TB drugs (Table 8).

This method also allowed for identification of heteroresistance by comparison of the relative number of reads for wild-type compared to the number of reads for mutants (Table 9). Heteroresistance was called when >15% and <80% mutant bases were detected.

TABLE 8

Example drug resistance profile of two samples sequenced using the developed method

| Sample | Ethambutol | Isoniazid | Pyrazinamide | Rifampicin | Streptomycin | Amikacin |
|---|---|---|---|---|---|---|
| 1 | Resistant | Resistant | Susceptible | Resistant | Susceptible | Resistant |
| 2 | Resistant | Resistant | Susceptible | Resistant | Resistant | Susceptible |
| 1 | Susceptible | Resistant | Susceptible | Susceptible | Susceptible | Resistant |
| 2 | Susceptible | Susceptible | Susceptible | Susceptible | Susceptible | Susceptible |
| 1 | | Susceptible | | Resistant | Resistant | Resistant |
| 2 | | Susceptible | | Susceptible | Susceptible | Resistant |

Table 9: Example heteroresistance detection results from two sequenced samples. Boxes with vertical stripes signify >80% of reads at that site are resistant associated mutants (resistant, no heteroresistance). Boxes with diagonal stripes signify 51%-79% of reads at that site are resistance associated mutants (heteroresistant, majority resistant bases). Black boxes signify 20%-50% of reads at that site are resistance associated mutants (heteroresistant, majority wild-type bases).

[image]

[image]

[image]

Raw read numbers could also be visualised, providing a more detailed analysis if required (Table 10). These results display the codon or nucleotide location within the annotated gene as well as the number of wild-type or mutant bases recorded at that location.

TABLE 10

Example of raw data provided through Epi2Me analysis for two sequenced samples

| Sample | Ethambutol Resistance SNP | Ethambutol Mutation | Ethambutol Wild-Type Bases | Ethambutol Mutant Bases |
|---|---|---|---|---|
| 1 | embB M306V | ATG -> GTG | 41 | 954 |
| 2 | embB M306I | ATG -> ATA | 45 | 662 |
| Sample | Isoniazid Resistance SNP | Isoniazid Mutation | Isoniazid Wild-Type Bases | Isoniazid Mutant Bases |
| 1 | katG S315T fabG1 T-8A | GCT -> GGT T->A | 35 50 | 2841 2929 |
| 2 | katG S315T | GCT -> GGT | 31 | 529 |
| Sample | Pyrazinamide Resistance SNP | Pyrazinamide Mutation | Pyrazinamide Wild-Type Bases | Pyrazinamide Mutant Bases |
| 1 | N/A | N/A | N/A | N/A |
| 2 | pncA V139A | CAC -> CGC | 865 | 507 |
| Sample | Rifampicin Resistance SNP | Rifampicin Mutation | Rifampicin Wild-Type Bases | Rifampicin Mutant Bases |
| 1 | rpoB D435G, rpoB L452P | GAC -> GGC CTG -> CCG | 148 73 | 1895 1629 |
| 2 | rpoB H445N, rpoB D435S (double mutation) | CAC -> AAC GAC -> TCC GAC -> TCC | 1396 1161 1462 | 1060 1385 758 |
| Sample | Streptomycin Resistance SNP | Streptomycin Mutation | Streptomycin Wild-Type Bases | Streptomycin Mutant Bases |
| 1 | N/A | N/A | N/A | N/A |
| 2 | gidB A205E rpsL K43R | TGC -> CGC AAG -> AGG | 18 52 | 1737 294 |
| 1 | rrs A1401G | A->G | 27 | 2908 |
| 2 | N/A | N/A | N/A | N/A |
| 1 | rrs A1401G | A->G | 27 | 2908 |
| 2 | N/A | N/A | N/A | N/A |
| 1 | N/A | N/A | N/A | N/A |
| 2 | gyrA D94G | GAC -> GGC | 3347 | 2004 |
| 1 | N/A | N/A | N/A | N/A |
| 2 | N/A | N/A | N/A | N/A |
| 1 | rrs A1401G | A->G | 27 | 2908 |
| 2 | N/A | N/A | N/A | N/A |
| 1 | gyrA A90V | GCG -> GTG | 331 | 3644 |
| 2 | gyrA D94G | GAC -> GGC | 3347 | 2004 |
| 1 | gyrA A90V | GCG -> GTG | 331 | 3644 |
| 2 | gyrA D94G | GAC -> GGC | 3347 | 2004 |
| 1 | gyrA A90V | GCG -> GTG | 331 | 3644 |
| 2 | gyrA D94G gyrA D89N | GAC -> GGC GAC -> AAC | 3347 2338 | 2004 3506 |

### Example 2

Following on from Example 1, a set of samples were processed with an altered DNA extraction and simplified library preparation method. Here, DNA was extracted instead using the Promega Maxwell RSC 48 with the PureFood Pathogen kit and within the library preparation alterations were made to the end-prep and barcode/adapter ligation reactions. The resistance profile was compared between methods to ensure the same profile was identified. Details of the method alterations are below:

DNA Extraction:
1. In a Microbiological Class II Safety Cabinet (MSC-II) in the level 3 containment facility (CL3) unseal liquid clinical sample and aliquot 750 µL to a fresh 1.5 mL Eppendorf tube with screw cap.
2. In MSC-II load sample Eppendorf tubes into an aerosol-sealable centrifuge rotor.
3. Centrifuge 750 µL clinical sputum sample at 15,000xg for 5 min, after which the centrifuge rotor is returned to the MSC-II and samples removed.
4. In MSC-II carefully remove supernatant and resuspend pellet in 700 µL Phosphate Buffered Saline (PBS).
5. In MSC-II transfer 700 µL of resuspended samples to bead-beating tubes with screw cap (Lysing Matrix E tubes from MP Biomedical).
6. In MSC-II bead-beat samples in a FastPrep-24 homogenizer at maximum speed for 45 seconds.
7. Repeat Step 6.
8. In MSC-II load bead-beating tubes into an aerosol-sealable centrifuge rotor.
9. Spin down bead-beating tubes at maximum speed for 3 minutes.
10. Return centrifuge rotor to the MSC-II and gently remove bead-beating tubes.
11. In MSC-II transfer 400 µL clear supernatant in two 200 µL aliquots to a clean 2 ml screw-capped sample tube. Add 40 µL Proteinase K to sample.
12. In MSC-II add 200 µL of Lysis Buffer A from the Maxwell RSC PureFood Pathogen Kit [Promega]
13. In MSC-II incubate samples on heat block for 10 minutes at 65° C. vortexing in the MSC-II every 30 seconds.
14. In MSC-II add 400 µL PBS and 300 µL Lysis Buffer from the Maxwell RSC PureFood Pathogen Kit [Promega]
15. Transfer samples to the Maxwell RSC sample well and prepare the automated extraction according to manufacturer instructions.
16. When automated extraction is completed return elution tubes to MSC-II for Multiplex PCR Preparation.

End Prep
1. Transfer 12.5 µL (< 450 ng) of pooled DNA to a thin-walled PCR plate
2. Add following reagents to the DNA

| Reagent | Volume per Sample (µL) |
|---|---|
| Ultra II End-Prep Buffer | 1.75 |
| Ultra II End-Prep Enzyme Mix | 0.75 |
| Total with DNA | 15 |

3. Mix by pipette
4. Spin down tube and incubate for 5 minutes at 20° C. followed by 5 minutes at 65° C.

Barcode Ligation
5. In a fresh 96-well plate add the following reagents in order per sample.
   a. 3 µL Nuclease-Free H₂O
   b. 0.75 µL End-Prepped DNA
   c. 1.25 µL Native Barcode (1 per Sample)
   d. 5 µL Blunt/TA Ligase Master Mix
6. Mix by pipetting and briefly spin down plate.
7. Incubate for 20 minutes at 20° C. followed by 10 minutes at 65° C.
8. Pool all samples in a clean 1.5 mL Eppendorf and carry 480 µL forward
   e. If pooled volume is <480 µL use total volume instead
9. Perform a 0.4x Bead Wash
   f. 192 µL of resuspended AMPure XP Beads for 480 µL of pooled sample
10. Incubate samples for 10 minutes at room temperature on a Hula Mixer
11. Place the sample on a magnet rack and incubate for 5 minutes
12. Carefully remove the supernatant and resuspend the bead pellet in 700 µL Short Fragment Buffer (SFB) [Oxford Nanopore]
13. Return the sample to the magnet rack and incubate for 5 minutes
14. Repeat steps 12 and 13
15. Carefully remove the supernatant and, leaving the tube on the magnet rack, wash the bead pellet with 100 µL 70% ethanol
16. Remove the supernatant and briefly spin down the tube before replacing it on the magnet rack
17. Using a p10 remove any residual supernatant and allow the pellet to air dry for approximately 30 seconds
   a. Take care not to let the pellet crack
18. Resuspend the pellet in 35 µL of nuclease-free H₂O and incubate for 2 minutes at room temperature
19. Return the tube to the magnet rack and incubate for 2 minutes, carefully transfer 35 µL of supernatant to a clean Eppendorf.

Adapter Ligation:
20. Thaw and spin down Adapter Mix (AMII) [ONT], Quick Ligation Reaction Buffer [NEB], Quick T4 Ligase [NEB], and Elution Buffer (EB) [ONT], and SFB [ONT]
21. Place thawed and vortexed reagents on ice
22. Mix the following in a 1.5 mL Eppendorf in order:

| Reagent | Volume (µL) |
|---|---|
| End-Prepped DNA | 30 |
| Quick Ligation Reaction Buffer | 10 |
| NEBNext Quick T4 DNA Ligase | 5 |
| Adapter Mix (AMII) | 5 |
| Total | 50 |

23. Gently mix tube by flicking and spin down
24. Incubate for 20 minutes at room temperature
25. Perform a 0.4x bead wash (20 µL) using resuspended AMPure XP beads
26. Incubate samples for 10 minutes on a hula mixer
27. Briefly spin down samples and place tube on magnet-rack and let incubate for 5 minutes
28. Carefully remove supernatant and resuspend the pellet in 125 µL SFB
29. Place tube on magnet-rack and let incubate for 5 minutes
30. Repeat steps 28 and 29
31. Briefly spin down tube and return to magnet-rack
32. Using a p10 remove residual supernatant
33. Air dry pellet for approximately 30 seconds
   a. Take care not to let the pellet crack
34. Resuspend pellet in 15 µL EB and incubate at room temperature for 10 minutes
35. Place tube on magnet-rack until elute is clear and colourless
36. Carefully remove and retain 15 µL eluted supernatant in clean 1.5 mL Eppendorf
37. Perform Qubit HS Assay on 1 µL elute.

Resistance to ‘first- and second-line anti-TB drugs was identified using the ONT Epi2Me FastQ TB Resistance Profile pipeline. Wild-type and mutant nucleotides were reported for all drug resistance associated SNP sites detected within the PCR product fastQ sequences. The presence of SNPs (>15% mutant bases) in specific target genes indicated resistance to specific anti-TB drugs (Table 11).

TABLE 11

Example drug resistance profile of two samples sequenced using the developed method

| Sample | Ethambutol | Isoniazid | Pyrazinamide | Rifampicin | Streptomycin | Amikacin |
|---|---|---|---|---|---|---|
| 1 | Resistant | Resistant | Susceptible | Resistant | Susceptible | Resistant |
| 2 | Resistant | Resistant | Susceptible | Resistant | Resistant | Susceptible |
| Sample | Bedaquiline | Capreomycin | Ciprofloxacin | Clofazimine | Ethionamide | Kanamycin |
| 1 | Susceptible | Resistant | Susceptible | Susceptible | Susceptible | Resistant |
| 2 | Susceptible | Susceptible | Susceptible | Susceptible | Susceptible | Susceptible |
| Sample | Linezolid | Moxifloxacin | Ofloxacian | Quinolones | | |
| 1 | Susceptible | Resistant | Resistant | | Resistant | |
| 2 | Susceptible | Susceptible | Susceptible | | Resistant | |

Raw read numbers could also be visualised, providing a more detailed analysis if required (Table 12) e.g. for identifying heteroresistance. These results display the codon or nucleotide location within the annotated gene as well as the number of wild-type or mutant bases recorded at that location.

TABLE 12

Example of raw data provided through Epi2Me analysis for two sequenced samples

| Sample | Ethambutol Resistance SNP | Ethambutol Mutation | Ethambutol Wild-Type Bases | Ethambutol Mutant Bases |
|---|---|---|---|---|
| 1 | embB M306V | ATG -> GTG | 41 | 954 |
| 2 | embB M306I | ATG -> ATA | 45 | 662 |

| | | | | |
|---|---|---|---|---|
| Sample | Isoniazid Resistance SNP | Isoniazid Mutation | Isoniazid Wild-Type Bases | Isoniazid Mutant Bases |
| 1 | katG S315T fabG1 T-8A | GCT-> GGT T->A | 35 50 | 2841 2929 |
| 2 | katG S315T | GCT -> GGT | 31 | 529 |

| Sample | Pyrazinamide Resistance SNP | Pyrazinamide Mutation | Pyrazinamide Wild-Type Bases | Pyrazinamide Mutant Bases |
|---|---|---|---|---|
| 1 | N/A | N/A | N/A | N/A |
| 2 | pncA V139A | CAC -> CGC | 865 | 507 |
| Sample | Rifampicin Resistance SNP | Rifampicin Mutation | Rifampicin Wild-Type Bases | Rifampicin Mutant Bases |
| 1 | rpoB D435G, rpoB L452P | GAC -> GGC CTG -> CCG | 148 73 | 1895 1629 |
| 2 | rpoB H445N, rpoB D435S (double mutation) | CAC -> AAC GAC -> TCC GAC -> TCC | 1396 1161 1462 | 1060 1385 758 |
| Sample | Streptomycin Resistance SNP | Streptomycin Mutation | Streptomycin Wild-Type Bases | Streptomycin Mutant Bases |
| 1 | N/A | N/A | N/A | N/A |
| 2 | gidB A205E rpsL K43R | TGC -> CGC AAG -> AGG | 18 52 | 1737 294 |
| Sample | Amikacin Resistance SNP | Amikacin Mutation | Amikacin Wild-Type Bases | Amikacin Mutant Bases |
| 1 | rrs A1401G | A->G | 27 | 2908 |
| 2 | N/A | N/A | N/A | N/A |
| Sample | Capreomycin Resistance SNP | Capreomycin Mutation | Capreomycin Wild-Type Bases | Capreomycin Mutant Bases |
| 1 | rrs A1401G | A->G | 27 | 2908 |
| 2 | N/A | N/A | N/A | N/A |
| Sample | Ciprofloxacin Resistance SNP | Ciprofloxacin Mutation | Ciprofloxacin Wild-Type Bases | Ciprofloxacin Mutant Bases |
| 1 | N/A | N/A | N/A | N/A |
| 2 | gyrA D94G | GAC -> GGC | 3347 | 2004 |
| Sample | Ethionamide Resistance SNP | Ethionamide Mutation | Ethionamide Wild-Type Bases | Ethionamide Mutant Bases |
| 1 | N/A | N/A | N/A | N/A |
| 2 | N/A | N/A | N/A | N/A |
| Sample | Kanamycin Resistance SNP | Kanamycin Mutation | Kanamycin Wild-Type Bases | Kanamycin Mutant Bases |
| 1 | rrs A1401G | A->G | 27 | 2908 |
| 2 | N/A | N/A | N/A | N/A |
| Sample | Moxifloxacin Resistance SNP | Moxifloxacin Mutation | Moxifloxacin Wild-Type Bases | Moxifloxacin Mutant Bases |
| 1 | gyrA A90V | GCG -> GTG | 331 | 3644 |
| 2 | gyrA D94G | GAC -> GGC | 3347 | 2004 |
| Sample | Ofloxacin Resistance SNP | Ofloxacin Mutation | Ofloxacin Wild-Type Bases | Ofloxacin Mutant Bases |
| 1 | gyrA A90V | GCG -> GTG | 331 | 3644 |
| 2 | gyrA D94G | GAC -> GGC | 3347 | 2004 |
| Sample | Quinolones Resistance SNP | Quinolones Mutation | Quinolones Wild-Type Bases | Quinolones Mutant Bases |
| 1 | gyrA A90V | GCG -> GTG | 331 | 3644 |
| 2 | gyrA D94G gyrA D89N | GAC -> GGC GAC -> AAC | 3347 2338 | 2004 3506 |

As can be seen from both results tables the alterations in methodology did not change the resistance profile of this sample. Therefore the optimised method (using the Promega Maxwell and simplified library preparation) would be the method of choice for this assay.

TABLE 13

Drug resistance profile of a sample sequenced using method 1 (Example 1) and 2 (Example 2)
Resistance call

| | | |
|---|---|---|
| Drug | Method 1 | Method 2 |
| Ethambutol | Resistant | Resistant |
| Isoniazid | Resistant | Resistant |
| Pyrazinamide | Resistant | Resistant |
| Rifampicin | Resistant | Resistant |
| Streptomycin | Resistant | Resistant |
| Amikacin | Susceptible | Susceptible |
| Capreomycin | Susceptible | Susceptible |
| Bedaquiline | Susceptible | Susceptible |
| Ciprofloxacin | Susceptible | Susceptible |
| Clofazamine | Susceptible | Susceptible |
| Ethionamide | Susceptible | Susceptible |
| Kanamycin | Susceptible | Susceptible |
| Linezolid | Susceptible | Susceptible |
| Moxifloxacin | Susceptible | Susceptible |
| Ofloxacin | Susceptible | Susceptible |
| Quinolones | Susceptible | Susceptible |

TABLE 14

Example of raw data provided through Epi2Me analysis for a sample comparing methods 1 (Example 1) and 2 (Example 2).

| Sample | Ethambutol Resistance SNP | Ethambutol Mutation | Ethambutol Wild-Type Bases | Ethambutol Mutant Bases |
|---|---|---|---|---|
| Method 1 | embB G406D embB E378A | GGC -> GAC GAG -> GCG | 115 23 | 303 379 |
| Method 2 | embB G406D embB E378A embB S347I | GGC -> GAC GAG -> GCG AGT -> GGT | 219 20 1004 | 1684 1814 306 |
| Sample | Isoniazid Resistance SNP | Isoniazid Mutation | Isoniazid Wild-Type Bases | Isoniazid Mutant Bases |
| Method 1 | katG S315T fabG1 C-15T | GCT -> GGT C->T | 8 38 | 281 1604 |
| Method 2 | katG S315T fabG1 C-15T | GCT -> GGT C->T | 51 12 | 5440 2526 |
| Sample | Pyrazinamide Resistance SNP | Pyrazinamide Mutation | Pyrazinamide Wild-Type Bases | Pyrazinamide Mutant Bases |
| Method 1 | pncA C14. | GCA -> TCA | 42 | 737 |
| Method 2 | pncA C14. | GCA -> TCA | 66 | 3208 |
| Sample | Rifampicin Resistance SNP | Rifampicin Mutation | Rifampicin Wild-Type Bases | Rifampicin Mutant Bases |
| Method 1 | rpoB H445C (double mutation) | CAC -> TGC | 248 141 | 1378 1407 |
| Method 2 | rpoB H445C (double mutation) | CAC -> TGC | 298 144 | 1613 2628 |
| Sample | Streptomycin Resistance SNP | Streptomycin Mutation | Streptomycin Wild-Type Bases | Streptomycin Mutant Bases |
| Method 1 | gidB A205E | TGC -> CGC | 17 | 888 |
| Method 2 | gidB A205E | TGC -> CGC | 28 | 3311 |

### REFERENCES

1. Coscolla M, Gagneux S. Seminars in Immunology Consequences of genomic diversity in Mycobacterium tuberculosis. Semin. Immunol. 2014;26(6):431-444. Available at http://dx.doi.org/10.1016/j.smim.2014.09.012.

2. Doughty EL, Sergeant MJ, Adetifa I, Antonio M, Pallen MJ. Culture-independent detection and characterisation of Mycobacterium tuberculosis and M. africanum in sputum samples using shotgun metagenomics on a benchtop sequencer. PeerJ. 2014;2:1-18.

3. Chatterjee A, Nilgiriwala K, Saranath D, Rodrigues C, Mistry N. Whole genome sequencing of clinical strains of Mycobacterium tuberculosis from Mumbai, India : A potential tool for determining drug-resistance and strain lineage. Tuberculosis. 2017;107:63-72. Available at https://doi.org/10.1016/j.tube.2017.08.002.

4. Costa P, Botelho A, Couto I, Viveiros M, Inácio J. Standing of nucleic acid testing strategies in veterinary diagnosis laboratories to uncover Mycobacterium tuberculosis complex members. Front. Mol. Biosci. 2014;1 (October):1-11.

5. Gupta S, Kakkar V. Biosensors and Bioelectronics Recent technological advancements in tuberculosis diagnostics - A review. Biosens. Bioelectron. 2018;115(May):14-29. Available at https://doi.org/10.1016/j.bios.2018.05.017.

6. Wlodarska M, Johnston JC, Gardy JL. A Microbiological Revolution Meets an Ancient Disease : Improving the Management of Tuberculosis with Genomics. 2015;28(2):523-539.

7. Jagielski T, Minias A, Ingen J Van, Rastogi N, Brzostek A. Methodological and Clinical Aspects of the Molecular Epidemiology of Mycobacterium tuberculosis and Other Mycobacteria. Clin. Microbiol. Rev. 2016;29(2):239-290.

8. N′Dira Sanoussi C, Affolabi D, Rigouts L, Anagonou S, Jong B de. Genotypic characterization directly applied to sputum improves the detection of Mycobacterium africanum West African 1, under-represented in positive cultures. PL₀S Negl. Trop. Dis. 2017:1-13.

9. Rue-albrecht K, Magee DA, Killick KE, et al. Comparative functional genomics and the bovine macrophage response to strains of the Mycobacterium genus. Front. Immunol. 2014;5(November):1-14.

10. IngenJ Van, Rahim Z, Mulder A, et al. Characterization of Mycobacterium orygis as M tuberculosis Complex Subspecies. Emerg. Infect. Dis. 2012;18(4):653--655.

11. Dippenaar A, David S, Parsons C, et al. Whole genome sequence analysis of Mycobacterium suricattae. Tuberculosis. 2015;95(6):682-688. Available at http://dx.doi.org/10.1016/j.tube.2015.10.001.

12. Alexander KA, Laver PN, Williams MC, et al. Pathology of the Emerging Mycobacterium tuberculosis Complex Pathogen, Mycobacterium mungi, in the Banded Mongoose ( Mungos mungo ). 2018;55(2):303-309.

13. Guthrie JL, Gardy JL. A brief primer on genomic epidemiology: lessons learned from Mycobacterium tuberculosis. Ann. N. Y. Acad. Sci. 2016:59-78.

14. Mcnerney R, Clark TG, Campino S, et al. International Journal of Infectious Diseases Removing the bottleneck in whole genome sequencing of Mycobacterium tuberculosis for rapid drug resistance analysis: a call to action. Int. J. Infect. Dis. 2017;56:130-135. Available at http://dx.doi.org/10.1016/j.ijid.2016.11.422.

15. Pankhurst LJ, Elias O, Votintseva AA, et al. Rapid, comprehensive, and aff ordable mycobacterial diagnosis with whole-genome sequencing: a prospective study. Lancet Respir. 4(1):49-58. Available at http://dx.doi.org/10.1016/S2213-2600(15)00466-X.

16. Brown AC, Bryant JM, Einer-jensen K, et al. Rapid Whole-Genome Sequencing of Mycobacterium tuberculosis Isolates Directly from Clinical Samples. J. Clin. Microbiol. 2015;53(7):2230-2237.

17. Kulchavenya E. Extrapulmonary tuberculosis: are statistical reports accurate? Ther. Adv. Infect. Dis. 2014;2(2):61-70.

18. Fisher M, Dolby T, Surtie S, et al. Improved method for collection of sputum for tuberculosis testing to ensure adequate sample volumes for molecular diagnostic testing. J. Microbiol. Methods. 2017;135:35-40. Available at: http://dx.doi.org/10.1016/j.mimet.2017.01.011.

19. World Health Organization. Global Tuberculosis Report. 2019.

20. Quan TP, Bawa Z, Foster D, et al. Evaluation of Whole-Genome Sequencing for Mycobacterial Species Identification and Drug Susceptibility Testing in a Clinical Setting: a Large-Scale Prospective Assessment of Performance against Line Probe Assays and Phenotyping. J. Chromatogr. B Anal. Technol. Biomed. Life Sci. 2018;56(2):1-14.

21. Zumla A, Al-Tawfiq JA, Enne VI, et al. Rapid point of care diagnostic tests for viral and bacterial respiratory tract infections-needs, advances, and future prospects. Lancet Infect. Dis. 2014;14(11):1123-1135.

22. Walker TM, Kohl TA, Omar S V, et al. Whole-genome sequencing for prediction of Mycobacterium tuberculosis drug susceptibility and resistance: a retrospective cohort study. Lancet Infect. Dis. 2015;15:1193-1202.

23. Gardy JL. Towards genomic prediction of drug resistance in tuberculosis. Lancet Infect. Dis. 2015;15(10):1124-1125. Available at http://dx.doi.org/10.1016/S1473-3099(15)00088-2.

24. Bradley P, Gordon NC, Walker TM, et al. Rapid antibiotic-resistance predictions from genome sequence data for Staphylococcus aureus and Mycobacterium tuberculosis. Nat. Commun. 2015;6:1-14. Available at http://dx.doi.org/10.1038/ncomms10063.

25. Papaventsis D, Casali N, Kontsevaya I, et al. Whole genome sequencing of Mycobacterium tuberculosis for detection of drug resistance : a systematic review. Clin. Microbiol. Infect. 2017;23(2):61-68. Available at: http://dx.doi.org/10.1016/j.cmi.2016.09.008.

26. Nimmo C, Doyle R, Burgess C, et al. International Journal of Infectious Diseases Rapid identi fi cation of a Mycobacterium tuberculosis full genetic drug resistance pro fi le through whole genome sequencing directly from sputum. Int. J. Infect Dis. 2017;62:44-46. Available at http://dx.doi.org/10.1016/j.ijid.2017.07.007.

27. Linger Y, Knickerbocker C, Sipes D, et al. Genotyping Multidrug-Resistant Mycobacterium tuberculosis from Primary Sputum and Decontaminated Sediment with an Integrated Microfluidic Amplification Microarray Test. J. Clin. Microbiol. 2018;56(3):1-11.

28. Miotto P, Tessema B, Tagliani E, et al. A standardised method for interpreting the association between mutations and phenotypic drug resistance in Mycobacterium tuberculosis. Eur. Respir. J. 2017;50. Available at: http://dx.doi.org/10.1183/13993003.01354-2017.

29. World Health Organization. The use of next-generation sequencing technologies for the detection of mutations associated with drug resistance in Mycobacterium tuberculosis complex: technical guide. 2018.

30. Votintseva AA, Bradley P, Pankhurst LJ, et al. Same-Day Diagnostic and Surveillance Data for Tuberculosis via Whole-Genome Sequencing of Direct Respiratory Samples. J. Clin. Microbiol. 2017;55(5):1285-1298.

31. Haas CT, Roe JK, Pollara G, Mehta M, Noursadeghi M. Diagnostic ‘omics’ for active tuberculosis. BMC Med. 2016. Available at: http://dx.doi.org/10.1186/s12916-016-0583-9.

32. Lee RS, Pai M. Real-Time Sequencing of Mycobacterium tuberculosis: Are We There Yet? J. Clin. Microbiol. 2017;55(5):1249-1254.

33. Allahyartorkaman M, Mirsaeidi M, Hamzehloo G, et al. Low diagnostic accuracy of Xpert MTB /RIF assay for extrapulmonary tuberculosis : A multicenter surveillance. Sci. Rep. 2019;9:1-6. Available at: http://dx.doi.org/10.1038/s41598-019-55112-y.

34. Jouet A, Gaudin C, Badalato N, et al. free prediction of susceptibility or resistance to 13 anti-tuberculous drugs. Eur. Respir. J. 2020;(June 2020). Available at http://dx.doi.org/10.1183/13993003.02338-2020.

35. Feuerriegel S, Kohl TA, Utpatel C, et al. Early View Rapid genomic first- and second-line drug resistance prediction from clinical Mycobacterium tuberculosis specimens using Deeplex ® -MycTB. Eur. Respir. J. 2020.

36. World Health Organization. The Use of Next-Generation Sequencing Technologies for the Detection of Mutations Associated with Drug Resistance in Mycobacterium tuberculosis Complex: Technical Guide. 2018.

37. Meier A, Kirschner P, Bange FC, Vogel U, Bottger EC. Genetic alterations in streptomycin-resistant Mycobacterium tuberculosis: Mapping of mutations conferring resistance. Antimicrob. Agents Chemother. 1994;38(2):228-233.

38. Karimi, S., Mirhendi, H., Zaniani F., Manesh, S., Salehi, M., Esfahani B. Rapid detection of streptomycin-resistant Mycobacterium tuberculosis by rpsL-restriction fragment length polymorphism. Adv. Biomed. Res. 2017;6(126).

39. Villellas C, Aristimuño L, Vitoria MA, et al. Analysis of mutations in streptomycin-resistant strains reveals a simple and reliable genetic marker for identification of the Mycobacterium tuberculosis Beijing genotype. J. Clin. Microbiol 2013;51(7):2124-2130.

40. Morlock GP, Metchock B, Sikes D, Crawford JT, Cooksey RC. ethA, inhA and katG Loci of ethionamide- resistant Clinical MTB isolates. Antimicrob. Agents Chemother. 2003;47(12):3799-3805.

41. Zhao L, Sun Q, Liu H, et al. Analysis of embCAB Mutations Associated with Ethambutol Resistance in Multidrug-Resistant Mycobacterium tuberculosis Isolates from China. Antimicrob. Agents Chemother. 2015;59(4):2045-2050.

42. Maus CE, Plikaytis BB, Shinnick TM. Mutation of tlyA confers capreomycin resistance in Mycobacterium tuberculosis. Antimicrob. Agents Chemother. 2005;49(2):571-7. Available at http://www.ncbi.nlm.nih.gov/pubmed/15673735%0Ahttp://www.pubmedcentral.nih.gov/articlere nder.fcgi?artid=PMC547314.

43. (NCBI) NC for BI. Mycobacterium tuberculosis. Available at https://www.ncbi.nlm.nih.gov/genome/?term=h37rv [Accessed Jul. 17, 2020].

TABLE 5

Optimisation testing results for primer design versions 1-48 in Multiplex measured by nested qPCR

| Multiplex Primer Design Version | eis CT | emBCT | fabG1CT | rrs CT | rv0678 CT | ethA CT | gyrA CT | rp08 CT | rplc CT | katG CT | hsp65 CT | pncA CT | inhA CT | gidB CT | tlyA CT | rpsL CT | rrl CT |
|---|---|---|---|---|---|---|---|---|---|---|---|---|---|---|---|---|---|
| 1* | 6 | 15.57 | 5 | 5 | 6 | 5 | 6 | 5 | 5 | 5 | N/A | 6 | 5 | 5 | 5 | 5 | 5 |
| 2* | 9.73 | 19.77 | 8.68 | 9.27 | 9.98 | 8.82 | 10.56 | 9.31 | 9.8 | 9.75 | N/A | 11.38 | 8.62 | 10.34 | 8.99 | 10.04 | 9.09 |
| 3* | 15.12 | 22.75 | 10.55 | 8.91 | 7.76 | 7.38 | 8.1 | 8.5 | 7.25 | 7.77 | 9.13 | 8.6 | 8.19 | 7.99 | 7.22 | 8.55 | 7.91 |
| 4* | 17.43 | 14.86 | 11.49 | 13.75 | 9.63 | 8.01 | 11.06 | 10.52 | 10.42 | 8.6 | 9.22 | 9.65 | 8.75 | 9.5 | 12.39 | 10.71 | 8.4 |
| 5* | 18.94 | 19.77 | 9.8 | 11.76 | 10.33 | 9.35 | 9.23 | 10.22 | 9.58 | 10.99 | 10.86 | 10.85 | 10.02 | 8.98 | 10.53 | 6.69 | 9.93 |
| 6* | 17.73 | 24.28 | 10.63 | 10.95 | 8.84 | 7.7 | 10.64 | 10.88 | 11.08 | 10.61 | 11.67 | 11.26 | 9.62 | 10.27 | 11.66 | 9.57 | 9.92 |
| 7* | 13.51 | 6 | 7.48 | 8.2 | 9.9 | N/A | N/A | N/A | N/A | N/A | N/A | N/A | N/A | N/A | N/A | N/A | N/A |
| 8* | 14.77 | 5 | 5 | 5 | 5 | N/A | N/A | N/A | N/A | N/A | N/A | N/A | N/A | N/A | N/A | N/A | N/A |
| 9* | 13.67 | 95 | 7.66 | 5 | 5 | N/A | N/A | N/A | N/A | N/A | N/A | N/A | N/A | N/A | N/A | N/A | N/A |
| 10* | 35 | 19.81 | 6 | 35 | 6.84 | N/A | N/A | N/A | N/A | N/A | N/A | N/A | N/A | N/A | N/A | N/A | N/A |
| 11* | 20.07 | 6 | 5 | 5 | 35 | N/A | N/A | N/A | N/A | N/A | N/A | N/A | N/A | N/A | N/A | N/A | N/A |
| 12* | 14.6 | 7.6 | 6.58 | 6 | 6 | N/A | N/A | N/A | N/A | N/A | N/A | N/A | N/A | N/A | N/A | N/A | N/A |
| 13* | 15.62. | 8.87 | 8.76 | 6.84 | 7.45 | N/A | N/A | N/A | N/A | N/A | N/A | N/A | N/A | N/A | N/A | N/A | N/A |
| 14* | 15.01 | 35 | 9.1 | 7.02 | 7.77 | N/A | N/A | N/A | N/A | N/A | N/A | N/A | N/A | N/A | N/A | N/A | N/A |
| 15* | 15.33 | 9.58 | 9.67 | 6.4 | 7.91 | N/A | N/A | N/A | N/A | N/A | N/A | N/A | N/A | N/A | N/A | N/A | N/A |
| 16* | 14.06 | 9.48 | 9.66 | 7.51 | 7.15 | N/A | N/A | N/A | N/A | N/A | N/A | N/A | N/A | N/A | N/A | N/A | N/A |
| 17* | 15.6 | 9.53 | 10.14 | 7.8 | 8.02 | N/A | N/A | N/A | N/A | N/A | N/A | N/A | N/A | N/A | N/A | N/A | N/A |
| 18* | 16.87 | 8.83 | 9.58 | 6.72 | 7.05 | N/A | N/A | N/A | N/A | N/A | N/A | N/A | N/A | N/A | N/A | N/A | N/A |
| 19* | 14.46 | 9.43 | 9.77 | 7.64 | 6.03 | N/A | N/A | N/A | N/A | N/A | N/A | N/A | NA | N/A | N/A | N/A | N/A |
| 20* | 14.36 | 9.61 | 9.73 | 7.45 | 7.55 | N/A | N/A | N/A | N/A | N/A | N/A | N/A | N/A | N/A | N/A | N/A | N/A |
| 21* | 13.67 | 9.51 | 9.14 | 7.38 | 7.63 | N/A | N/A | N/A | N/A | N/A | N/A | N/A | N/A | N/A | N/A | N/A | N/A |
| 22* | 15.07 | 12.7 | 8.98 | 6.99 | 7.97 | N/A | N/A | N/A | N/A | N/A | N/A | N/A | N/A | N/A | N/A | N/A | N/A |
| 23* | N/A | N/A | N/A | N/A | N/A | 9.91 | 12.94 | 14.02 | 10.66 | 11.62 | 11.27 | N/A | N/A | N/A | N/A | N/A | N/A |
| 24* | N/A | N/A | N/A | N/A | N/A | 13.61 | 13.35 | 11.19 | 12.64 | 11.85 | 11.54 | N/A | N/A | N/A | N/A | N/A | N/A |
| 25* | N/A | N/A | N/A | N/A | N/A | 12.62 | 11.96 | 12.11 | 11.55 | 11.47 | 11.78 | N/A | N/A | N/A | N/A | N/A | N/A |
| 26* | N/A | N/A | N/A | N/A | N/A | 10.84 | 12 | 12.09 | 11.96 | 10.63 | 11.13 | N/A | N/A | N/A | N/A | N/A | N/A |
| 27* | N/A | N/A | N/A | N/A | N/A | 12.29 | 12.02 | 12.75 | 29.5 | 11.27 | 11.29 | N/A | N/A | N/A | N/A | N/A | N/A |
| 28* | N/A | N/A | N/A | N/A | N/A | 8.43 | 13.76 | 11.92 | 9.75 | 17.77 | 10.45 | N/A | N/A | N/A | N/A | N/A | N/A |
| 29* | N/A | N/A | N/A | N/A | N/A | 9.6 | 11.33 | 12.27 | 18.26 | 10.3 | 11.53 | N/A | N/A | N/A | N/A | N/A | N/A |
| Table 5 Continued | | | | | | | | | | | | | | | | | |
| 30* | N/A | N/A | N/A | N/A | N/A | 9.34 | 10.91 | 9.57 | 8.49 | 13.65 | 9.69 | N/A | N/A | N/A | N/A | N/A | N/A |
| 31* | N/A | N/A | N/A | N/A | N/A | 8.74 | 12.11 | 10.69 | 10.23 | 18.95 | 10.99 | N/A | N/A | N/A | N/A | N/A_{.} | N/A |
| 32* | N/A | N/A | N/A_{.} | N/A | N/A | 9.04 | 11.77 | 11.61 | 11.66 | 15.08 | 11.6 | N/A_{.} | N/A | N/A | N/A | N/A | N/A |
| 33* | N/A | N/A | N/A | N/A_{.} | N/A | N/A | N/A | N/A | N/A | N/A | N/A | 11.65 | 9.17 | 13.11 | 16.09 | 29.26 | 11.76 |
| 34* | N/A | N/A_{.} | N/A_{.} | N/A | N/A_{.} | N/A | N/A | N/A | N/A | N/A | N/A | 12.53 | 9.64 | 10.35 | 18.54 | 11.84 | 19.14 |
| 35* | N/A_{.} | N/A | N/A_{.} | N/A | N/A_{.} | N/A | N/A | N/A | N/A | N/A_{.} | N/A | 11.47 | 9.6 | 10.34 | 14.63 | 29.11 | 11.73 |
| 36* | N/A | N/A_{.} | N/A | N/A | N/A | N/A | N/A | N/A | N/A | N/A | N/A | 12.88 | 9.29 | 11.44 | 17.33 | 13.31 | 13.53 |
| 37* | N/A | N/A_{.} | N/A | N/A | N/A | N/A | N/A | N/A | N/A | N/A | N/A | 14.01 | 9.92 | 11.69 | 18.26 | 28.95 | 12.84 |
| 38* | N/A_{.} | N/A | N/A | N/A | N/A | 16.58 | 17.47 | 17.12 | 16.01 | 40 | 28 | N/A | N/A | N/A | N/A_{.} | N/A | N/A |
| 39* | N/A | N/A | N/A | N/A | N/A | 28 | 40 | 40 | 28 | 19.7 | 28 | N/A_{.} | N/A | N/A | N/A | N/A | N/A |
| 40* | N/A | N/A_{.} | N/A | N/A | N/A_{.} | 28 | 18.87 | 18.98 | 28 | 17.67 | 21.74 | N/A_{.} | N/A | N/A | N/A | N/A | N/A |
| 41* | N/A | N/A_{.} | N/A_{.} | N/A | N/A | 40 | 40 | 40 | 16.86 | 28 | 28 | N/A_{.} | N/A_{.} | N/A_{.} | N/A_{.} | N/A | N/A |
| 42* | N/A_{.} | N/A | N/A | N/A | N/A_{.} | 21.88 | 17.93 | 18.01 | 40 | 28 | 28 | N/A | N/A_{.} | N/A_{.} | N/A | N/A | N/A |
| 43* | N/A_{.} | N/A_{.} | N/A | N/A N/A | N/A | 18.03 | 17.3 | 40 | 13.57 | 16.05 | 22.87 | N/A_{.} | N/A | N/A | N/A | N/A | N/A |
| 44* | N/A_{.} | N/A | N/A_{.} | N/A | N/A_{.} | 40 | 40 | 40 | 40 | 40 | N/A | N/A_{.} | N/A | N/A | N/A_{.} | N/A_{.} | N/A |
| 45* | N/A | N/A_{.} | N/A | N/A | N/A | 40 | 40 | 40 | 40 | 40 | N/A | N/A_{.} | N/A_{.} | N/A_{.} | N/A | N/A | N/A |
| 46* | N/A | N/A | N/A | N/A | N/A_{.} | 40 | 40 | 40 | 40 | 40 | N/A | N/A | N/A_{.} | N/A | N/A_{.} | N/A | N/A |
| 47* | N/A_{.} | N/A | N/A | N/A | N/A | 40 | 40 | 40 | 40 | 40 | N/A | N/A | N/A | N/A_{.} | N/A_{.} | N/A | N/A |
| 48* | N/A_{.} | N/A_{.} | N/A | N/A | N/A_{.} | 34.62 | 15.06 | 15.92 | 13.87 | 14.6 | N/A | N/A_{.} | N/A_{.} | N/A | N/A_{.} | N/A_{.} | N/A |

◆Nested qPCR performed with undiluted multiplex product May skew results due to extremely early florescense. [symbol]
Design changes occurred only in Multiplex Group 1. Groups 2 and 3 remained unchanged during this period. [symbol]
Design changes occurred only in Multiplex Group 2. Groups 1 and 3 remained unchanged during this period. [symbol]
Design changes occurred only in Multiplex Group 3. Groups 1 and 2 remained unchanged ed during this period.

TABLE A

| rpoB | |
|---|---|
| Codon | |
| 170 | Valine to Phenylalanine |
| 286 | Alanine to Valine |
| 359 | Valine to Alanine |
| 400 | Threonine to Alanine |
| 424 | Phenylalanine to Leucine |
| 424 | Phenylalanine to Serine |
| 424 | Phenylalanine to Valine |
| 425 | Phenylalanine Deletion |
| 426 | Glycine Deletion |
| 427 | Threonine Deletion |
| 428 | Serine Deletion |
| 429 | Glutamine Deletion |
| 430 | Leucine Deletion |
| 431 | Serine to Threonine |
| 432 | Glutamine Deletion |
| 432 | Glutamine to Histidine |
| 432 | Glutamine to Lysine |
| 432 | Glutamine to Leucine |
| 432 | Glutamine to Proline |
| 433 | Phenylalanine Deletion |
| 433 | Phenylalanine Duplication |
| 434 | Methionine Deletion |
| 434 | Methionine to Isoleucine |
| 435 | Aspartic Acid Deletion |
| 435 | Aspartic acid to Tyrosine |
| 435 | Aspartic acid to Alanine |
| 435 | Aspartic acid to Glycine |
| 435 | Aspartic acid to insertion |
| 435 | Aspartic acid to Asparagine |
| 435 | Aspartic acid to Valine |
| 436 | Glutamine Deletion |
| 437 | Asparagine Deletion |
| 438 | Asparagine Deletion |
| 439 | Proline Deletion |
| 440 | Leucine Deletion |
| 441 | Serine Deletion |
| 441 | Serine to Glutamine |
| 442 | Glycine Deletion |
| 443 | Leucine Deletion |
| 444 | Threonine Deletion |
| 445 | Histidine Deletion |
| 445 | Histidine to Cysteine |
| 445 | Histidine to Aspartic acid |
| 445 | Histidine to Phenylalanine |
| 445 | Histidine to Glycine |
| 445 | Histidine to Leucine |
| 445 | Histidine to Arginine |
| 445 | Histidine to Tyrosine |
| 446 | Lysine Deletion |
| 447 | Arginine Deletion |
| 448 | Arginine Deletion |
| 449 | Leucine Deletion |
| 450 | Serine to Leucine |
| 450 | Serine to Phenylalanine |
| 450 | Serine to Leucine |
| 450 | Serine to Glutamine |
| 450 | Serine to Tryptophan |
| 450 | Serine to Tyrosine |
| 451 | Alanine Deletion |
| 452 | Leucine Deletion |
| 452 | Leucine to Proline |
| 454 | Proline to Histidine |
| 454 | Proline to Leucine |
| 460 | Glutamic Acid to Glycine |
| 480 | Isoleucine to Threonine |
| 480 | Isoleucine to Valine |
| 491 | Isoleucine to Phenylalanine |
| 493 | Serine to Leucine |
| 513 | Glutamine to Lysine |
| 513 | Glutamine to Leucine |
| 513 | Glutamine to Proline |
| 514 | Phenylalanine duplicate |
| 516 | Aspartic Acid to Alanine |
| 516 | Aspartic Acid to Phenylalanine |
| 516 | Aspartic Acid to Glycine |
| 516 | Aspartic Acid to Valine |
| 516 | Aspartic Acid to Tyrosine |
| 518 | Asparagine deletion |
| 522 | Serine to Leucine |
| 526 | Histidine to Cysteine |
| 526 | Histidine to Proline |
| 526 | Histidine to Aspartic Acid |
| 526 | Histidine to Glycine |
| 526 | Histidine to Leucine |
| 526 | Histidine to Arginine |
| 526 | Histidine to Tyrosine |
| 531 | Serine to Phenylalanine |
| 531 | Serine to Leucine |
| 531 | Serine to Tryptophan |
| 533 | Leucine to Proline |
| 40 | Threonine to Isoleucine |
| 43 | Lysine Deletion |
| 43 | Lysine to Arginine |
| 43 | Lysine to Threonine |
| 88 | Lysine Deletion |
| 88 | Lysine to Glutamine |
| 88 | Lysine to Arginine |
| -83 | C to T |
| 7 | C to T |
| 26 | Frameshift |
| 52 | C to T |
| 64 | C to T |
| 200 | CtoA |
| 353 | T to C |
| 383 | T to A |
| 397 | C insertion Frameshift |
| 555 | T to G |
| 758 | Frameshift |
| 236 | Asparagine to Lysine |
| 63 | Serine to Arginine |
| -8 | T Deletion |
| -15 | C Deletion |
| -15 | C to T |
| -16 | A Deletion |
| -17 | GtoT |
| 70 | Histidine to Arginine |
| 74 | Alanine to Serine |
| 85 | Histidine Deletion |
| 86 | Proline Deletion |
| 87 | Histidine Deletion |
| 88 | Glycine to Cysteine |
| 88 | Glycine Deletion |
| 89 | Aspartic Acid to Asparagine |
| 89 | Aspartic Acid Deletion |
| 90 | Alanine to Valine |
| 90 | Alanine Deletion |
| 91 | Serine to Proline |
| 91 | Serine Deletion |
| 92 | Isoleucine Deletion |
| 93 | Tyrosine Deletion |
| 94 | Aspartic Acid to Alanine |
| 94 | Aspartic Acid to Glycine |
| 94 | Aspartic Acid to Asparagine |
| 94 | Aspartic Acid to Histidine |
| 94 | Aspartic Acid Deletion |
| 96 | Leucine Deletion |
| 97 | Valine Deletion |
| -14 | C to T |
| -10 | G to A |
| 296 | Asparagine to Histidine |
| 297 | Serine to Alanine |
| 306 | Methionine Deletion |
| 313 | Alanine to Valine |
| 319 | Tyrosine to Cysteine |
| 319 | Tyrosine to Serine |
| 328 | Aspartic Acid to Glycine |
| 328 | Aspartic Acid to Valine |
| 328 | Aspartic Acid to Tyrosine |
| 334 | Tyrosine to Histidine |
| 347 | Serine to Isoleucine |
| 354 | Aspartic Acid to Alanine |
| 356 | Alanine to Valine |
| 377 | Valine to Glycine |
| 378 | Glutamic Acid to Alanine |
| 397 | Proline to Threonine |
| 405 | Glutamic Acid to Aspartic Acid |
| 406 | Glycine to Alanine |
| 406 | Glycine to Cysteine |
| 406 | Glycine to Aspartic Acid |
| 406 | Glycine to Serine |
| 497 | Glutamine to Lysine |
| 497 | Glutamine to Proline |
| 497 | Glutamine to Arginine |
| 504 | Glutamic Acid to Aspartic Acid |
| 905 | CtoA |
| 905 | C to G |
| 906 | A to G |
| 907 | A to C |
| 907 | A to T |
| 908 | A to G |
| 1239 | T to C |
| 1325 | A to C |
| 1338 | A to C |
| 1401 | A to G |
| 1401 | A Deletion |
| 1402 | C to T |
| 1402 | C Deletion |
| 1484 | G to Deletion |
| 1484 | GtoT |
| 1 | Methionine to Arginine |
| 21 | Isoleucine to Threonine |
| 21 | Isoleucine to Valine |
| 43 | Glycine to Cysteine |
| 61 | Threonine to Methionine |
| 232 | Threonine to Alanine |
| 338 | Isoleucine to Serine |
| 342 | Threonine to Lysine |
| 381 | Alanine to Proline |
| 154 | Cysteine to Arginine |
| 155 | Tyrosine to Cysteine |
| 155 | Tyrosine to Serine |
| 159 | Leucine to Proline |
| 180 | Threonine to Lysine |
| 182 | Glycine to Arginine |
| 191 | Tryptophan to Glycine |
| 191 | Tryptophan to Arginine |
| 232 | Proline to Arginine |
| 257 | Methionine to Isoleucine |
| 275 | Threonine to Alanine |
| 295 | Glutamine to Proline |
| 297 | Glycine to Valine |
| 299 | Glycine to Cysteine |
| 300 | Tryptophan to Cysteine |
| 300 | Tryptophan to Serine |
| 302 | Serine to Arginine |
| 311 | Aspartic Acid to Glycine |
| 315 | Serine to Isoleucine |
| 315 | Serine to Asparagine |
| 315 | Serine to Threonine |
| 315 | Serine deletion |
| 321 | Tryptophan to Stop Codon |
| 328 | Tryptophan to Leucine |
| 335 | Isoleucine to Valine |
| 378 | Leucine to Proline |
| 379 | Alanine to Valine |
| 419 | Aspartic Acid to Histidine |
| 424 | Alanine to Glycine |
| 11 | Isoleucine to Asparagine |
| 19 | Alanine to Proline |
| 26 | Leucine to Phenylalanine |
| 30 | Glycine to Aspartic Acid |
| 34 | Glutamine to Valine |
| 41 | Valine to Isoleucine |
| 47 | Arginine to Tryptophan |
| 48 | Histidine to Asparagine |
| 48 | Histidine to Glutamine |
| 52 | Cysteine to Phenylalanine |
| 64 | Arginine to Tryptophan |
| 65 | Valine to Glycine |
| 69 | Glutamine to Aspartic Acid |
| 70 | Serine to Asparagine |
| 73 | Glycine to Alanine |
| 75 | Proline to Leucine |
| 75 | Proline to Arginine |
| 79 | Leucine to Serine |
| 79 | Leucine to Tryptophan |
| 80 | Alanine to Proline |
| 83 | Arginine to Proline |
| 85 | Aspartic Acid to Alanine |
| 88 | Valine to Alanine |
| 91 | Leucine to Proline |
| 92 | Glutamic Acid to Aspartic Acid |
| 93 | Proline to Leucine |
| 117 | Glycine to Valine |
| 118 | Arginine to Leucine |
| 118 | Arginine to Serine |
| 125 | Glutamine to Stop Codon |
| 134 | Alanine to Glutamic Acid |
| 136 | Serine to Stop Codon |
| 137 | Arginine to Proline |
| 137 | Arginine to Tryptophan |
| 138 | Alanine to Threonine |
| 138 | Alanine to Valine |
| 149 | Serine to Arginine |
| 162 | Isoleucine to Serine |
| 173 | Glutamic Acid to Stop Codon |
| 195 | Tyrosine to Histidine |
| 200 | Alanine to Glutamic Acid |
| 203 | Valine to Leucine |
| 205 | Alanine to Glutamic Acid |
| -12 | T to C |
| -11 | A to G |
| -7 | T to C |
| 1 | Methionine to Threonine |
| 3 | Alanine to Glutamic Acid |
| 4 | Leucine to Serine |
| 6 | Isoleucine to Threonine |
| 7 | Valine to Glycine |
| 8 | Aspartic Acid to Glycine |
| 8 | Aspartic Acid to Asparagine |
| 8 | Aspartic Acid to Glutamic Acid |
| 9 | Valine to Alanine |
| 10 | Glutamine to Arginine |
| 10 | Glutamine to Proline |
| 10 | Glutamine deletion |
| 12 | Aspartic Acid to Alanine |
| 12 | Aspartic Acid to Asparagine |
| 14 | Cysteine to Arginine |
| 14 | Cysteine deletion |
| 14 | Cysteine to Glycine |
| 14 | Cysteine to Tyrosine |
| 17 | Glycine to Aspartic Acid |
| 19 | Leucine to Proline |
| 21 | Valine to Glycine |
| 24 | Glycine to Aspartic Acid |
| 27 | Leucine to Proline |
| 32 | Serine to Isoleucine |
| 34 | Tyrosine deletion |
| 34 | Tyrosine to Aspartic Acid |
| 35 | Leucine to Arginine |
| 46 | Alanine to Valine |
| 46 | Alanine to Glutamic Acid |
| 47 | Threonine to Alanine |
| 47 | Threonine to Proline |
| 48 | Lysine to Threonine |
| 49 | Aspartic Acid to Alanine |
| 49 | Aspartic Acid to Glycine |
| 49 | Aspartic Acid to Asparagine |
| 51 | Histidine to Glutamine |
| 51 | Histidine to Arginine |
| 51 | Histidine to Tyrosine |
| 54 | Proline to Serine |
| 54 | Proline to Leucine |
| 57 | Histidine to Aspartic Acid |
| 57 | Histidine to Proline |
| 57 | Histidine to Arginine |
| 57 | Histidine to Tyrosine |
| 58 | Phenylalanine to Leucine |
| 58 | Phenylalanine to Serine |
| 59 | Serine to Proline |
| 61 | Threonine to Proline |
| 62 | Proline to Glutamine |
| 62 | Proline to Leucine |
| 63 | Aspartic Acid to Glycine |
| 63 | Aspartic Acid to Alanine |
| 64 | Tyrosine to Aspartic Acid |
| 66 | Serine to Proline |
| 67 | Serine to Proline |
| 68 | Tryptophan to Cysteine |
| 68 | Tryptophan to Arginine |
| 68 | Tryptophan to Glycine |
| 69 | Proline to Leucine |
| 71 | Histidine to Tyrosine |
| 71 | Histidine to Glutamine |
| 71 | Histidine to Arginine |
| 71 | Histidine to Aspartic Acid |
| 72 | Cysteine to Arginine |
| 72 | Cysteine to Tyrosine |
| 76 | Threonine to Proline |
| 76 | Threonine to Isoleucine |
| 78 | Glycine to Cysteine |
| 78 | Glycine to Aspartic Acid |
| 81 | Phenylalanine to Valine |
| 82 | Histidine to Arginine |
| 82 | Histidine to Aspartic Acid |
| 85 | Leucine to Proline |
| 85 | Leucine to Arginine |
| 87 | Threonine to Methionine |
| 90 | Isoleucine to Serine |
| 94 | Phenylalanine to Leucine |
| 94 | Phenylalanine to Serine |
| 96 | Lysine to Asparagine |
| 96 | Lysine to Arginine |
| 96 | Lysine to Glutamic Acid |
| 96 | Lysine to Threonine |
| 97 | Glycine to Aspartic Acid |
| 97 | Glycine to Cysteine |
| 97 | Glycine to Serine |
| 99 | Tyrosine deletion |
| 102 | Alanine to Valine |
| 103 | Tyrosine duplication |
| 103 | Tyrosine deletion |
| 103 | Tyrosine to Histidine |
| 104 | Serine to Arginine |
| 104 | Serine to Glycine |
| 108 | Glycine to Arginine |
| 114 | Threonine to Proline |
| 116 | Leucine to Proline |
| 116 | Leucine to Arginine |
| 120 | Leucine to Proline |
| 123 | Arginine to Proline |
| 125 | Valine to Phenylalanine |
| 125 | Valine to Glycine |
| 128 | Valine to Glycine |
| 130 | Valine to Glycine |
| 132 | Glycine to Alanine |
| 132 | Glycine to Aspartic Acid |
| 132 | Glycine to Serine |
| 133 | Isoleucine to Threonine |
| 134 | Alanine to Valine |
| 135 | Threonine to Proline |
| 135 | Threonine to Asparagine |
| 137 | Histidine to Proline |
| 137 | Histidine to Arginine |
| 138 | Cysteine to Arginine |
| 138 | Cysteine to Serine |
| 138 | Cysteine to Tyrosine |
| 139 | Valine to Glycine |
| 139 | Valine to Leucine |
| 139 | Valine to Alanine |
| 139 | Valine to Methionine |
| 141 | Glutamine to Proline |
| 141 | Glutamine deletion |
| 142 | Threonine to Alanine |
| 142 | Threonine to Lysine |
| 142 | Threonine to Methionine |
| 146 | Alanine to Threonine |
| 146 | Alanine to Valine |
| 148 | Indel Arginine insert (in frame) |
| 151 | Leucine to Serine |
| 154 | Arginine to Glycine |
| 155 | Valine to Glycine |
| 155 | Valine to Alanine |
| 155 | Valine to Leucine |
| 159 | Leucine to Valine |
| 159 | Leucine to Proline |
| 160 | Threonine to Proline |
| 161 | Alanine to Proline |
| 162 | Glycine to Aspartic Acid |
| 168 | Threonine to Proline |
| 171 | Alanine to Glutamic Acid |
| 172 | Leucine to Proline |
| 175 | Methionine to Threonine |
| 175 | Methionine to Valine |
| 180 | Valine to Phenylalanine |
| 180 | Valine to Glycine |
| 2058 | G Deletion |
| -15 | C to T |
| 21 | Isoleucine to Threonine |
| 21 | Isoleucine to Valine |
| 49 | Serine to Alanine |
| 194 | Isoleucine to Threonine |

1. One or more oligonucleotide primer sets for amplifying a portion of one or more genes from M. tuberculosis and/or related bacteria in the M. tuberculosis complex selected from the group comprising one or more of eis, embB, ethA, fabG1, gidB, gyrA, inhA, katG, pncA, rrl, rplC, rpoB, rpsL, rrs, rv0678 and tlyA, wherein each set comprises a pair of forward and reverse primers specific for said portion, wherein each primer has a sequence as set out in SEQ ID Nos. 1-32.

2. One or more oligonucleotide primer sets as claimed in claim 1 for use in multiplex PCR, wherein the sets of primers are grouped into one or more multiplex groups, wherein the multiplex groups comprise forward and reverse primer pairs for amplifying a portion of:
(a) eis, embB, rrs, rv0678, and fabG1;
(b) gyrA, rpoB, ethA, rplC, and katG; and/or
(c) gidB, inhA, rrl, pncA, rpsL, and tlyA.

3. One or more oligonucleotide primer sets for use in multiplex PCR and grouped into one or more multiplex groups as claimed in claim 1, wherein the one or more multiplex groups comprise:
(a) one or more of SEQ ID Nos. 1 and 2; 3 and 4; 5 and 6; 7 and 8; and 9 and 10 (Group 1 in Table 7);
(b) one or more of SEQ ID Nos. 11 and 12; 13 and 14; 15 and 16; 17 and 18; and 19 and 20 (Group 2 in Table 7); and/or
(c) one or more of SEQ ID Nos. 21 and 22; 23 and 24; 25 and 26; 27 and 28; 29 and 30; and 31 and 32 (Group 3 in Table 7).

4. An oligonucleotide primer set group for use in multiplex PCR as claimed in claim 3 consisting of SEQ ID Nos. 1 and 2; 3 and 4; 5 and 6; 7 and 8; and 9 and 10 (Group 1 in Table 7).

5. An oligonucleotide primer set group for use in multiplex PCR as claimed in claim 3 consisting of SEQ ID Nos. 11 and 12; 13 and 14; 15 and 16; 17 and 18; and 19 and 20 (Group 2 in Table 7).

6. An oligonucleotide primer set group for use in multiplex PCR as claimed in claim 3 consisting of SEQ ID Nos. 21 and 22; 23 and 24; 25 and 26; 27 and 28; 29 and 30; and 31 and 32 (Group 3 in Table 7).

7. One or more oligonucleotide primer sets or oligonucleotide primer set groups as claimed in claim 1, wherein the portion of the one or more genes contains one or more mutations that confer antibiotic resistance to one or more of ethambutol, isoniazid, pyrazinamide, rifampicin, streptomycin, amikacin, bedaquiline, capreomycin, ciprofloxacin, clofazimine, ethionamide, kanamycin, linezolid, moxifloxacin, ofloxacin and quinoloes, preferably wherein the one or mutations are one or more single nucleotide polymorphisms.

8. A multiplex PCR reaction mixture comprising one or more groups of oligonucleotide primer sets for amplifying a portion of one or more genes from M. tuberculosis and/or related bacteria in the M. tuberculosis complex selected from the group comprising or consisting of one or more of eis, embB, ethA, fabG1, gidB, gyrA, inhA, katG, pncA, rrl, rplC, rpoB, rpsL, rrs, rv0678, tlyA, wherein each set comprises a pair of forward and reverse primers specific for said portion, wherein the groups of oligonucleotide primer sets comprise one or more of SEQ ID Nos. 1, 2, 3, 4, 5, 6, 7, 8, 9 and 10 (Group 1 in Table 7); one or more of SEQ ID Nos. 11, 12, 13, 14, 15, 16, 17, 18, 19 and 20 (Group 2 in Table 7); and/or one or more of SEQ ID Nos. 21, 22, 23, 24, 25, 26, 27, 28, 29, 30, 31 and 32 (Group 3 in Table 7).

9. A multiplex PCR reaction mixture as claimed in claim 8 comprising a group of oligonucleotide primer sets consisting of:
(a) SEQ ID Nos. 1, 2, 3, 4, 5, 6, 7, 8, 9 and 10 (Group 1 in Table 7);
(b) SEQ ID Nos. 11, 12, 13, 14, 15, 16, 17, 18, 19 and 20 (Group 2 in Table 7); or
(c) SEQ ID Nos. 21, 22, 23, 24, 25, 26, 27, 28, 29, 30, 31 and 32 (Group 3 in Table 7).

10. A method of detecting and/or identifying the presence of one or more mutations that confer antibiotic resistance in a sample comprising DNA from Mycobacterium tuberculosis and/or related bacteria in the M. tuberculosis complex, said method including the steps of:
(a) isolating or extracting DNA from the sample;
(b) amplifying relevant gene regions or amplicons by multiplex polymerase chain reaction using one or more groups of oligonucleotide primer sets as claimed in claim 2;
(c) subjecting the amplified gene regions or amplicons to DNA sequencing; and
detecting one or more mutations.

11. A method of predicting whether a patient suffering from tuberculosis will respond to treatment with one or more of ethambutol, isoniazid, pyrazinamide, rifampicin, streptomycin, amikacin, bedaquiline, capreomycin, ciprofloxacin, clofazimine, ethionamide, kanamycin, linezolid, moxifloxacin, ofloxacin and quinolones, said method comprising a step of determining the presence of one or more drug resistant mutations in one or more genes selected from the group comprising one or more of eis, embB, ethA, fabG1, gidB, gyrA, inhA, katG, pncA, rrl, rplC, rpoB, rpsL, rrs, rv0678 and tlyA in DNA obtained from a sample from the patient, the method comprising:
(a) isolating or extracting DNA from the sample;
(b) amplifying relevant gene regions or amplicons by multiplex polymerase chain reaction using one or more groups of oligonucleotide primer sets as claimed in claim 2;
(c) subjecting the amplified gene regions or amplicons to DNA sequencing; and
detecting the one or more mutations.

12. A method as claimed in claim 11, wherein:
(a) the method is for predicting whether a patient suffering from tuberculosis will respond to treatment with one or more of ethambutol, isoniazid, streptomycin, amikacin, bedaquiline, capreomycin, clofazimine, ethionamide, kanamycin, and the one or more genes are eis, embB, rrs, rv0678, and fabG1; and the group of oligonucleotide primer sets consists of SEQ ID Nos. 1, 2, 3, 4, 5, 6, 7, 8, 9 and 10 (Group 1 in Table 7);
(b) the method is for predicting whether a patient suffering from tuberculosis will respond to treatment with one or more of isoniazid, rifampicin, ciprofloxacin, ethionamide, linezolid, moxifloxacin, ofloxacin and quinolones whereupon the one or more genes are gyrA, rpoB, ethA, rplC, and katG; and the group of oligonucleotide primer sets consists of SEQ ID Nos. 11, 12, 13, 14, 15, 16, 17, 18, 19 and 20 (Group 2 in Table 7); and/or
(c) the method is for predicting whether a patient suffering from tuberculosis will respond to treatment with one or more of pyrazinamide, streptomycin, capreomycin and ethionamide whereupon the one or more genes are gidB, inhA, rrl, pncA, rpsL, and tlyA; and the group of oligonucleotide primer sets consists of SEQ ID Nos. 21, 22, 23, 24, 25, 26, 27, 28, 29, 30, 31 and 32 (Group 3 in Table 7).

13. A method as claimed in claim 10, wherein the sample is one or more tissues and/or bodily fluids obtained from a subject suspected of having, or confirmed to have TB, optionally wherein the sample is sputum; urine; blood; plasma; serum; synovial fluid; pus; cerebrospinal fluid; pleural fluid; pericardial fluid; ascitic fluid; sweat; saliva; tears; vaginal fluid; semen; interstitial fluid; bronchoalveolar lavage; bronchial wash; gastric lavage; gastric wash; a transtracheal or transbronchial fine needle aspiration; bone marrow; pleural tissue; tissue from a lymph node, mediastinoscopy, thoracoscopy or transbronchial biopsy; or combinations thereof; or a culture specimen of one or more tissues and/or bodily fluids obtained from a subject suspected of having or confirmed to have TB.

14. A method as claimed in claim 10, wherein when more than one group of primer oligonucleotide primer sets are used for the amplification step (step (b)), each group is run as a separate multiplex group template, preferably wherein one or more of the multiplex group templates are then pooled prior to step (c) to make a single template for DNA sequencing and mutation detection.

15. A method for determining an appropriate antibiotic treatment regime for a patient with tuberculosis, comprising detecting and/or identifying the presence of one or more mutations that confer antibiotic resistance in a sample from the subject using the method as claimed in claim 10, and determining an appropriate antibiotic regime on the basis of the mutations detected/identified.

16. A kit comprising one or more oligonucleotide primer sets or oligonucleotide primer set groups as claimed in claim 1,or a multiplex PCR reaction mixture.


SEQUENCE LISTING (33 sequences)

SEQ ID NO: 1 - DNA, 19 bp, Artificial Sequence
  eis Forward Primer
tgtcgggtacctttcgagc

SEQ ID NO: 2 - DNA, 19 bp, Artificial Sequence
  eis Reverse Primer
tccatgtacagcgccatcc

SEQ ID NO: 3 - DNA, 19 bp, Artificial Sequence
  embB Forward Primer
cgccgtggtgatattcggc

SEQ ID NO: 4 - DNA, 19 bp, Artificial Sequence
  embB Reverse Primer
gcacaccgtagctggagac

SEQ ID NO: 5 - DNA, 20 bp, Artificial Sequence
  rrs Forward Primer
ctctgggcagtaactgacgc

SEQ ID NO: 6 - DNA, 19 bp, Artificial Sequence
  rrs Reverse Primer
gagtgttgcctcaggaccc

SEQ ID NO: 7 - DNA, 19 bp, Artificial Sequence
  rv0678 Forward Primer
gctcgtccttcacttcgcc

SEQ ID NO: 8 - DNA, 19 bp, Artificial Sequence
  rv0678 Reverse Primer
atcagtcgtcctctccggt

SEQ ID NO: 9 - DNA, 19 bp, Artificial Sequence
  fabG1 Forward Primer
cttttgcacgcaattgcgc

SEQ ID NO: 10 - DNA, 19 bp, Artificial Sequence
  fabG1 Reverse Primer
agcagtcctgtcatgtgcg

SEQ ID NO: 11 - DNA, 19 bp, Artificial Sequence
  gyrA Forward Primer
tgacagacacgacgttgcc

SEQ ID NO: 12 - DNA, 19 bp, Artificial Sequence
  gyrA Reverse Primer
cgatcgctagcatgttggc

SEQ ID NO: 13 - DNA, 20 bp, Artificial Sequence
  rpoB Forward Primer
tcatcatcaacgggaccgag

SEQ ID NO: 14 - DNA, 20 bp, Artificial Sequence
  rpoB Reverse Primer
acacgatctcgtcgctaacc

SEQ ID NO: 15 - DNA, 19 bp, Artificial Sequence
  ethA Forward Primer
tggatccatgaccgagcac

SEQ ID NO: 16 - DNA, 19 bp, Artificial Sequence
  eth A Reverse Primer
gtccaggaggcattggtgt

SEQ ID NO: 17 - DNA, 19 bp, Artificial Sequence
  rplC Forward Primer
agtacaaggactcgcggga

SEQ ID NO: 18 - DNA, 18 bp, Artificial Sequence
  rplC Reverse Primer
tcgagtgggtaccctggc

SEQ ID NO: 19 - DNA, 19 bp, Artificial Sequence
  KatG Forward Primer
ctgtggccggtcaagaaga

SEQ ID NO: 20 - DNA, 21 bp, Artificial Sequence
  KatG Reverse Primer redesigned
ggatctggctcttaaggctgg

SEQ ID NO: 21 - DNA, 19 bp, Artificial Sequence
  gidB Forward Primer
tgacacagacctcacgagc

SEQ ID NO: 22 - DNA, 19 bp, Artificial Sequence
  gidB Reverse Primer
gcccttctgattcgcgatg

SEQ ID NO: 23 - DNA, 19 bp, Artificial Sequence
  inhA Forward Primer
gggcgctgcaatttatccc

SEQ ID NO: 24 - DNA, 19 bp, Artificial Sequence
  inhA Reverse Primer
ggcgtagatgatgtcaccc

SEQ ID NO: 25 - DNA, 17 bp, Artificial Sequence
  rrl Forward Primer
ggtccgtgcgaagtcgc

SEQ ID NO: 26 - DNA, 18 bp, Artificial Sequence
  rrl Reverse Primer
tgaacccgtgttctgcgg

SEQ ID NO: 27 - DNA, 19 bp, Artificial Sequence
  pncA Forward Primer
tcaccggacggatttgtcg

SEQ ID NO: 28 - DNA, 19 bp, Artificial Sequence
  pncA Reverse Primer
tccagatcgcgatggaacg

SEQ ID NO: 29 - DNA, 18 bp, Artificial Sequence
  rpsL Forward Primer
gcggcgggtattgtggtt

SEQ ID NO: 30 - DNA, 18 bp, Artificial Sequence
  rpsL Reverse Primer
taaccggcgcttctcacc

SEQ ID NO: 31 - DNA, 18 bp, Artificial Sequence
  tlyA Forward Primer
cgttgatgcgcagcgatc

SEQ ID NO: 32 - DNA, 18 bp, Artificial Sequence
  tlyA Reverse Primer
ggtctcggtggcttcgtc

SEQ ID NO: 33 - DNA, 19 bp, Artificial Sequence
  KatG Reverse Primer initial
tgcccggatctggctctta